(19) United States
(12) Patent Application Publication
(10) Pub. No.: US 2012/0295797 A1
(43) Pub. Date: Nov. 22, 2012

(54) METHODS FOR DIAGNOSIS AND PROGNOSIS OF PULMONARY HYPERTENSION

(76) Inventors: Peter L. Jones, Philadelphia, PA (US); Darren Taichman, Narberth, PA (US)

(21) Appl. No.: 12/936,389

(22) PCT Filed: Apr. 2, 2009
(86) PCT No.: PCT/US09/02050

§ 371 Date: Sep. 26, 2011

Related U.S. Application Data
(60) Provisional application No. 61/064,912, filed on Apr. 2, 2008.

(51) Int. Cl. C40B 30/04; G01N 33/566; C12Q 1/68

(52) U.S. Cl. 506/9; 435/6.12; 436/501; 435/6.11; 435/7.92

(57) ABSTRACT

The invention relates to diagnostic and prognostic assays and kits for pulmonary hypertension (PH) and types thereof. The invention includes detecting the expression level of markers associated with pulmonary hypertension for diagnosis, progosis, or treatment of pulmonary hypertension.

### CROSS REFERENCE TO RELATED APPLICATIONS

This application claims priority to U.S. Provisional Patent Application 61/064,912, filed Apr. 2, 2008, which is incorporated herein by reference in its entirety.

### FIELD OF THE INVENTION

This invention is directed to diagnostic and prognostic assays and kits for pulmonary hypertension and types thereof.

### BACKGROUND OF THE INVENTION

The normal adult pulmonary circulation is a low-resistance system that allows high flow to be maintained by the right ventricle at relatively low pressures. In several distinct clinical disorders, however, pulmonary vascular resistance rises, resulting in pulmonary arterial hypertension (PAH) and an increase in the work demanded of the right ventricle to maintain cardiac output. As resistance and pressure continue to rise, there is progressive impairment in right heart function. The curtailed pulmonary vasculature and impaired cardiac output results in dyspnea and exercise limitations. Ultimately, the increased afterload results in right-sided heart failure and premature patient death. In the absence of effective therapy, the median survival of untreated patients with PAH is 2.8 years from the time of diagnosis. While remarkable progress in understanding the mechanisms of PAH has led to the development of new therapies, prognosis remains poor with up to 50% of patients dying within 5 years, despite the most aggressive treatments. Thus, new insights into the diagnosis, pathophysiology, and treatment of PAH are importantly needed.

At the structural level, PAH is accompanied by pulmonary artery (PA) vasoconstriction and/or vascular wall remodeling, together with reduced vascular distensibility. Remodeling of PAs in severe PAH may result from apoptosis, as well as proliferation of sub-sets of endothelial cells (ECs), and hypertrophy and hyperplasia of vascular smooth muscle cells (SMCs). In addition, expansion of the adventitial fibroblast layer, (together with its associated vasa vasorum via neo-angiogenesis), and recruitment of circulating inflammatory and EC precursors to the pulmonary vascular wall have been reported. Simultaneously, proteolysis and de novo synthesis of specific components of the extracellular matrix (ECM) occurs, further contributing to remodeling of the PA wall. Whether these and other changes within the pulmonary vascular wall are also reflected by alterations in the peripheral blood levels of specific circulating antigens is not well studied. Identification of such changes might help inform further studies investigating both basic pathobiologic mechanisms resulting in PAH, as well as aiding in the identification of new markers and therapeutic targets for this disease.

The vasculopathies in PAH are complex and may occur in the absence of identifiable risks, as in idiopathic pulmonary arterial hypertension (IPAH), or as a result of specific hereditary somatic mutations, (i.e. in the BMP type H receptor gene (BMPR2)), that occurs in certain forms of familial PAH (FPAH). In addition, associated forms of PAH (APAH) may occur with chronic liver disease (portopulmonary PAH), HIV infection, anorectic agent use, certain congenital heart diseases, or collagen vascular disease. Given the multiple origins of PAH, it is reasonable to hypothesize that therapies that benefit one patient would not hold the same promise for another patient. Indeed, differences in the response to individual therapies are seen not only in comparing patients with distinct forms of PAH, but also among patients with the same clinical diagnosis. For example, only a minority of patients with IPAH demonstrate significant, acute pulmonary vasoreactivity and sustained clinical response with Ca²⁺ channel antagonists. Overall, patients with scleroderma associated PAH do not experience the same benefit to various therapies as patients with IPAH. Clues to the underlying reasons for these differences might be aided by tissue biopsy. Biopsy, however, is accompanied by significant and often prohibitive risk in the setting of pulmonary hypertention, and is thus seldom feasible or pursued. Alternatively, less invasive means of evaluating differences between patients that might reflect the pathological events taking place, or their stage, would help to guide diagnosis or therapeutic decisions that would greatly enhance care.

Current therapy for pulmonary hypertension is inadequate. Vasodilators such as calcium channel antagonists are effective in only a small subpopulation of patients with IPAH (previously referred to as, primary pulmonary hypertension) and is often complicated by systemic hypotensive responses. Although prostenoid therapies are beneficial in improving the status of some patients, they require cumbersome and in some cases risky delivery systems and fail to provide a cure. Similarly, neither the endothelin receptor antagonists nor the phosphodiesterase inhibitors provide clinical improvement for all patients, and neither class of drug therapy is curative. Thus, despite current medical therapies, PAH remains progressive and life threatening with a significant reduction in survival. Heart-lung and single lung transplantation have been used on patients which do not respond to medical therapies, however, due to surgical morbidity and mortality, this approach is usually limited to those patients who continue to deteriorate despite aggressive therapy at centers experienced in management of this disease. Patients frequently die of right heart failure and those individuals which have signs of right heart failure have a mean survival of 6-12 months.

### SUMMARY OF THE INVENTION

In one embodiment, the invention provides a method for diagnosis of pulmonary hypertension, in a subject, comprising the steps of: obtaining a biological sample from said subject; and testing said biological sample to determine whether or not one or more markers is over-expressed or under-expressed in said biological sample, relative to the expression of said one or more markers in a control sample, whereby the over-expression or under-expression of said one or more of markers in said biological sample indicates said pulmonary hypertension, and whereby said one or more markers comprises at least one of the markers set forth in Table 5. In an exemplary embodiment, said method is a multi-analyte profiling method.

In another embodiment, the invention provides a method for detecting markers associated with pulmonary hypertension, in a subject, comprising the steps of: obtaining a biological sample from said subject; and testing said biological sample to determine whether or not one or more of markers is over-expressed or under-expressed in said biological sample, relative to the expression of said one or more of markers in a control sample, whereby the over-expression or under-expression of said one or more of markers in said biological sample indicates said pulmonary hypertension, and whereby said one or more of markers comprises at least one of the markers set forth in Table 5.

In another embodiment, the invention provides a method for predicting the progression of pulmonary hypertension, in a subject, comprising the steps of: obtaining a biological sample from said subject; and testing said biological sample to determine whether or not one or more of markers is over-expressed or under-expressed in said biological sample, relative to the expression of said one or more of markers in a control sample, whereby the over-expression or under-expression of said one or more of markers in said biological sample indicates the progression of said pulmonary hypertension, and whereby said one or more of markers comprises at least one of the markers set forth in Table 5.

In another embodiment, the invention provides a method of providing prognosis for pulmonary hypertension, in a subject, comprising the steps of: obtaining a biological sample from said subject; and testing said biological sample to determine whether or not one or more of markers is over-expressed or under-expressed in said biological sample, relative to the expression of said one or more of markers in a control sample, whereby the over-expression or under-expression of said one or more of markers in said biological sample indicates said pulmonary hypertension, and whereby said one or more of markers comprises at least one of the markers set forth in Table 5.

In another embodiment, the invention provides a method of screening a library of compounds to identify a compound to treat pulmonary hypertension, comprising the steps of: testing a biological sample to determine whether or not one or more of markers is over-expressed or under-expressed in said biological sample, relative to the expression of said one or more of markers in a control sample, whereby said one or more of markers comprises at least one of the markers set forth in Table 5, thereby screening said library of compounds.

In another embodiment, the invention provides a kit for diagnosis of pulmonary hypertension, comprising: antibodies that bind to one or more markers set forth in Table 5; and optionally, reagents for the formation of the medium favorable to the immunological reaction.

In another embodiment, the invention provides a kit for diagnosis of pulmonary hypertension, comprising: oligonucleotides complementary to one or more markers set forth in Table 5; and optionally, reagents for the hybridization between said one or more of oligonucleotides and said markers.

In another embodiment, the invention provides a kit to monitor a response to a therapy for pulmonary hypertension, comprising: antibodies that bind to one or more markers set forth in Table 5; and optionally, reagents for the formation of the medium favorable to the immunological reaction.

In another embodiment, the invention provides a kit to monitor a response to a therapy for pulmonary hypertension, comprising: oligonucleotides complementary to one or more markers set forth in Table 5; and optionally, reagents for the hybridization between said oligonucleotides and said markers.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings.

FIG. 1 shows that fold-increases for TN-C in IPAH patient plasma were the same using either MAP analysis or the sandwich ELISA (p=0.428). The boundary of the box closest to zero indicates the 25th percentile and the boundary of the box farthest from zero indicates the 75th percentile. A solid line within the box marks the median, and the dotted line marks the mean. Whiskers (error bars) above and below the box indicate the 90th and 10th percentiles. Open circles above and below the error bars indicate the 95^{th} and 5^{th} percentile respectively.

FIG. 2 shows selected specific antigens that were detected at significantly higher (n=35) or lower (n=4) levels in all PAH patient versus healthy controls. Endothelin-1 (ET-1) (A) and tumor necrosis factor-α (TNF-α) (B), MMP-9 (C) and tenascin-C (TN-C) (D), FGF-2 (E) MCP-1, EN-RAGE, IL-1ra, Cancer Antigen-125 and Cancer Antigen 19-9 (F-J). IL-4 (K) and IL-15 (L), were found to be significantly increased or decreased in both IPAH and NIPAH patients. Multiple cancer-related and/or angiogenic factors, including MMP-2, MMP-9 (C), TN-C (D), FGF-2 (E), VEGF (M), adiponectin, growth hormone, fibrinogen, and PAI-1 (Table 2) are upregulated significantly in the plasma of PAH patients. Erythropoietin (N), Serum amyloid P (O), IGF-1 (P). The boundary of the box closest to zero indicates the 25th percentile and the boundary of the box farthest from zero indicates the 75th percentile. A solid line within the box marks the median, and the dotted line marks the mean. Whiskers (error bars) above and below the box indicate the 90th and 10th percentiles. Open circles above and below the error bars indicate the 95^{th} and 5^{th} percentile respectively.

### DETAILED DESCRIPTION OF THE INVENTION

The invention relates to diagnostic and prognostic assays and kits for pulmonary hypertension and types thereof. Specifically, the invention relates to detecting the expression level of markers associated with pulmonary hypertension for diagnosis, progosis, and treatment of pulmonary hypertension.

In one embodiment, provided herein is a method for diagnosis of pulmonary hypertension, in a subject, comprising the steps of: obtaining a biological sample from said subject; and testing said biological sample to determine whether or not one or more of markers is over-expressed or under-expressed in said biological sample, relative to the expression of said one or more of markers in a control sample, whereby the over-expression or under-expression of said one or more of markers in said biological sample indicates said pulmonary hypertension, and whereby said one or more of markers comprises at least one of the markers set forth in Table 5. In another embodiment, provided herein is a method for detecting markers associated with pulmonary hypertension, in a subject, comprising the steps of: obtaining a biological sample from said subject; and testing said biological sample to determine whether or not one or more of markers is over-expressed or under-expressed in said biological sample, relative to the expression of said one or more of markers in a control sample, whereby the over-expression or under-expression of said one or more of markers in said biological sample indicates said pulmonary hypertension, and whereby said one or more of markers comprises at least one of the markers set forth in Table 5.

In another embodiment, provided herein is a method for predicting the progression of pulmonary hypertension, in a subject, comprising the steps of: obtaining a biological sample from said subject; and testing said biological sample to determine whether or not one or more of markers is over-expressed or under-expressed in said biological sample, relative to the expression of said one or more of markers in a control sample, whereby the over-expression or under-expression of said one or more of markers in said biological sample indicates the progression of said pulmonary hypertension, and whereby said one or more of markers comprises at least one of the markers set forth in Table 5. In another embodiment, provided herein is a method of providing prognosis for pulmonary hypertension, in a subject, comprising the steps of: obtaining a biological sample from said subject; and testing said biological sample to determine whether or not one or more of markers is over-expressed or under-expressed in said biological sample, relative to the expression of said one or more of markers in a control sample, whereby the over-expression or under-expression of said one or more of markers in said biological sample indicates said pulmonary hypertension, and whereby said one or more of markers comprises at least one of the markers set forth in Table 5.

In one embodiment, provided herein is a method for diagnosis of pulmonary hypertension, in a subject, comprising the steps of: obtaining a biological sample from said subject; and testing said biological sample to determine whether or not a plurality of markers is over-expressed or under-expressed in said biological sample, relative to the expression of said plurality of markers in a control sample, whereby the over-expression or under-expression of said plurality of markers in said biological sample indicates said pulmonary hypertension, and whereby said plurality of markers comprises one or more markers set forth in Table 5. In another embodiment, provided herein is a method for detecting markers associated with pulmonary hypertension, in a subject, comprising the steps of: obtaining a biological sample from said subject; and testing said biological sample to determine whether or not a plurality of markers is over-expressed or under-expressed in said biological sample, relative to the expression of said plurality of markers in a control sample, whereby the over-expression or under-expression of said plurality of markers in said biological sample indicates said pulmonary hypertension, and whereby said plurality of markers comprises one or more markers set forth in Table 5.

In another embodiment, provided herein is a method for predicting the progression of pulmonary hypertension, in a subject, comprising the steps of: obtaining a biological sample from said subject; and testing said biological sample to determine whether or not a plurality of markers is over-expressed or under-expressed in said biological sample, relative to the expression of said plurality of markers in a control sample, whereby the over-expression or under-expression of said plurality of markers in said biological sample indicates the progression of said pulmonary hypertension, and whereby said plurality of markers comprises one or more markers set forth in Table 5. In another embodiment, provided herein is a method of providing prognosis for pulmonary hypertension, in a subject, comprising the steps of: obtaining a biological sample from said subject; and testing said biological sample to determine whether or not a plurality of markers is over-expressed or under-expressed in said biological sample, relative to the expression of said plurality of markers in a control sample, whereby the over-expression or under-expression of said plurality of markers in said biological sample indicates said pulmonary hypertension, and whereby said plurality of markers comprises one or more markers set forth in Table 5.

The biological sample can be a tissue, blood, or other biological sample known to one of skill in the art. In one example, a tissue sample can be removed from a subject in accordance with a method known to one of skill in the art. In another example, a blood sample can be removed from a subject, and white blood cells can be isolated for extraction of nucleic acids by standard techniques.

In one embodiment, a control sample is obtained from a subject whose is healthy and free from a pulmonory hypertension. The expression level of a protein or a nucleic acid of a marker in the control sample is determined, and in one embodiment, such expression level serves as a control expression level for the marker. The expression level of a protein or a nucleic acid of a marker in a test sample obtained from a treatment subject, relative to its expression level in the control sample, is indicative of a pulmonory hypertension.

In one embodiment, the expression level of a protein or a nucleic acid of a marker in a test sample is greater than the expression level of the corresponding protein or nucleic acid in a control sample (i.e., expression of the marker is “over-expressed”). As used herein, the expression of a marker is “over-expressed” when the amount of its protein or nucleic acid expression in a test sample from a subject is greater than the amount of the expression level of the correponding protein or nucleic acid in a control sample. In another embodiment, the expression level of a protein or a nucleic acid of a marker in a test sample is less than the expression level of the corresponding protein or nucleic acid in a control sample (i.e., expression of the marker is “under-expressed”). As used herein, the expression of a marker is “under-expressed” when the amount of its protein or nucleic acid expression in a test sample from a subject is less than the amount of the expression level of the corresponding protein or nucleic acid in a control sample. The relative protein or nucleic acid expression in the control and normal samples can be determined with respect to one or more expression standards known in the art.

In some embodiments, markers of the invention are detected by binding of a capture molecule specific for the protein (for example, an aptamer, or an antibody in an immunoassay). The present invention is not limited to a particular capture molecule or antibody. Any capture molecule or antibody (e.g., monoclonal or polyclonal) that detects a marker may be utilized.

In one embodiment, antibody binding is detected by techniques known in the art. For example, in some embodiments, antibody binding is detected using a suitable technique, including but not limited to, radio-immunoassay, ELISA (enzyme-linked immunosorbant assay), “sandwich” immunoassay, immunoradiometric assay, gel diffusion precipitation reaction, immunodiffusion assay, precipitation reaction, agglutination assay (e.g., gel agglutination assay, hemagglutination assay, etc.), complement fixation assay, immunofluorescence assay, protein A assay, and immunoelectrophoresis assay.

In some embodiments, a quantitative ELISA assay is utilized (See e.g., U.S. Pat. Nos. 5,958,715, and 5,484,707, each of which is herein incorporated by reference). In some preferred embodiments, the quantitative ELISA is a competitive ELISA. The sample to be tested is added to the plate along with an antibody. The sample competes for binding to the antibody with the immobilized peptide. The plate is washed and the antibody bound to the immobilized protein is then detected using any suitable method (e.g., a secondary antibody comprising a label or a group reactive with an enzymatic detection system). The amount of signal may be inversely proportional to the amount of protein. In a particular embodiment, a multi-analyte plasma proteomics is used to detect markers of the invention. The multi-analyte plasma proteomics is well known in the art. An example of the multi-analyte plasma proteomics is described in the Example section.

In some embodiments, an automated detection assay is utilized. Methods for the automation of immunoassays include, but are not limited to, those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a diagnosis and/or prognosis based on the level of marker polypeptide in the plasma is utilized. In other embodiments, the immunoassay described in U.S. Pat. Nos. 5,789,261, 5,599,677 and 5,672,480, each of which is herein incorporated by reference, is utilized. In a preferred embodiment, a high-throughput detection assay is used.

In still other embodiments, a protein microarray or protein chip array assay is utilized for detection (See e.g., U.S. Pat. No. 6,197,599, herein incorporated by reference). In such an assay, proteins (e.g., antibodies specific for a marker polypeptide) are immobilized on a solid support such as a chip. A sample is passed over the solid support. Bound polypeptides are then detected using any suitable method. In some embodiments, detection is via surface plasmon resonance (SPR) (See e.g., WO 90/05305, herein incorporated by reference). In SPR, a beam of light from a laser source is directed through a prism onto a biosensor consisting of a transparent substrate, usually glass, which has one external surface covered with a thin film of a noble metal, which in turn is covered with an organic film that interacts strongly with an analyte, such as a biological, biochemical or chemical substance. The organic film contains antibodies (e.g., specific for a marker polypeptide of the present invention), which can bind with an analyte in a sample to cause an increased thickness, which shifts the SPR angle. By either monitoring the position of the SPR angle, or the reflectivity at a fixed angle near the SPR angle, the presence or absence of an analyte in the sample can be detected.

In other embodiments, The PROTEINCHIP (Ciphergen Biosystems, Fremont, Calif.) is utilized for detection. The PROTEINCHIP system uses SELDI (Surface-Enhanced Laser Desorption/Ionization) technology to perform the separation, detection and analysis of proteins at the femtomole level directly from biological samples (See e.g., U.S. Pat. No. 6,294,790 and U.S. Patent Application US20010014461A1, each of which is herein incorporated by reference. In the PROTEINCHIP technology, proteins of interest (e.g., cytokine, cytokine-related compound or chemokine polypeptides) are captured on the PROTEINCHIP Array (e.g., via a bound antibody) directly from the original source material. The chip is washed to remove undesired materials and bound proteins are detected using SELDI.

In some embodiments, a cytometric bead array assay is used (Quantum Plex kit, Bangs Laboratories; Cytometric Bead Array kit, BD Biosciences). These systems allow for multiple analyte detection with small volume samples. In other embodiments, a Luminex bead assay is used.

In one embodiment, provided herein is a quantitative, multi-analyte profiling (MAP) method for identifying the levels of circulating plasma antigens within healthy volunteers. In another embodiment, provided herein is a highly quantitative, multi-analyte profiling (MAP) method for identifying the levels of circulating plasma antigens within healthy volunteers, in patients with pulmonary arterial hypertension (PAH), idiopathic pulmonary arterial hypertension (IPAH), familial pulmonary arterial hypertension (FPAH), and associated forms of familial pulmonary arterial hypertension (APAH). In another embodiment, FPAH and APAH subjects are designated as belonging to the non-IPAH (NIPAH) group. non-idiopathic PAH (NIPAH)). In another embodiment, the method is used to identify and quantify circulating proteomic signatures that are not only specific for PAH patients. In another embodiment, the method is used to identify and quantify circulating proteomic signatures for IPAH patients. In another embodiment, the method is used to identify and quantify circulating proteomic signatures from patients with NIPAH. In another embodiment, the method is used to identify and quantify circulating proteomic signatures that are specific for PAH patients. In another embodiment, the method of identifying and quantifying circulating proteomic signatures is used to distinguish between IPAH and NIPAH patients.

In another embodiment, multi-analyte profiling (MAP) analysis represents a highly quantitative and rapid method for simultaneously analyzing a large number of specific antigens using a very small volume of patient plasma. In another embodiment, analysis of circulating antigen levels within a collected biological sample, via MAP, yields results equivalent to an ELISA assay. In another embodiment, MAP yields results with greater efficiency and with a higher throughput capacity, than an ELISA assay. In another embodiment, MAP analysis represents an efficient and simple approach, not only for diagnosis, but for selecting patients that would benefit from a therapy, or as a means to monitor a patient's response to a particular therapy.

In another embodiment, the expression level of a marker is determined by measuring its nucleic acid expression (e.g., mRNA, miRNA, etc.). The level of a nucleic acid, for example, an mRNA of a marker in a sample can be measured using any technique that is suitable for detecting nucleic acid expression levels in a biological sample. Suitable techniques for determining nucleic acid expression levels in cells from a biological sample are well known to those of skill in the art. Examples of such techniques include, but are not limited to, Northern blot analysis, RT-PCR, microarrays, in situ hybridization. In a particular embodiment, a high-throughput system, for example, a microarray, is used to measure the expression level of a plurality of genes.

In one embodiment, the level of an mRNA is detected using Northern blot analysis. For example, total cellular RNA can be purified from cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters. The RNA is then immobilized on the filters by heating. Detection and quantification of specific RNA is accomplished using appropriately labeled DNA or RNA probes complementary to the RNA in question.

Suitable probes for Northern blot hybridization of a given mRNA can be produced from the nucleic acid sequences of the mRNA. Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11.

In one example, the nucleic acid probe can be labeled with, e.g., a radionuclide, such as 3H, 32P, 33P, 14C, or 35S; a heavy metal; or a ligand capable of functioning as a specific binding pair member for a labeled ligand (e.g., biotin, avidin or an antibody), a fluorescent molecule, a chemiluminescent molecule, or an enzyme. Probes can be labeled to high specific activity by nick translation, random priming, or other methods known to one of skill in the art. For example, by replacing preexisting nucleotides with highly radioactive nucleotides according to the nick translation method, it is known to prepare 32P-labeled nucleic acid probes with a specific activity well in excess of 10⁸ cpm/microgram. Autoradiographic detection of hybridization can then be performed by exposing hybridized filters to photographic film. Densitometric scanning of the photographic films exposed by the hybridized filters provides an accurate measurement of mRNA transcript levels. In another embodiment, mRNA gene transcript levels can be quantified by computerized imaging systems, such the Molecular Dynamics 400-B 2D Phosphorimager available from Amersham Biosciences, Piscataway, N.J.

In another embodiment, the random-primer method can be used to incorporate an analogue, for example, the dTTP analogue 5-(N-(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin, and antibodies (e.g., anti-biotin antibodies) coupled to fluorescent dyes or enzymes that produce color reactions.

The relative number of mRNA gene transcripts in cells can also be determined by reverse transcription of mRNA gene transcripts, followed by amplification of the reverse-transcribed transcripts by polymerase chain reaction (RT-PCR). The levels of mRNA gene transcripts can be quantified in comparison with an internal standard, for example, the level of mRNA from a “housekeeping” gene present in the same sample. A suitable “housekeeping” gene for use as an internal standard includes, e.g., myosin or glyceraldehyde-3-phosphate dehydrogenase (G3PDH). The methods for quantitative RT-PCR and variations thereof are within the skill in the art.

In another embodiment, an oligolibrary, in microchip format (i.e., a microarray), may be constructed containing a set of probe oligodeoxynucleotides that are specific for a set of mRNA genes. Using such a microarray, the expression level of multiple mRNAs in a biological sample can be determined by reverse transcribing the RNAs to generate a set of target oligodeoxynucleotides, and hybridizing them to probe oligodeoxynucleotides on the microarray to generate a hybridization, or expression, profile. The hybridization profile of the test sample can then be compared to the pre-determined expression level of a control sample to determine which mRNAs have an altered expression level. As used herein, “probe oligonucleotide” or “probe oligodeoxynucleotide” refers to an oligonucleotide that is capable of hybridizing to a target oligonucleotide. “Target oligonucleotide” or “target oligodeoxynucleotide” refers to a molecule to be detected (e.g., via hybridization).

An “expression profile” or “hybridization profile” of a particular sample may be a fingerprint of the state of the sample; while two states may have any particular gene similarly expressed, the evaluation of a number of genes simultaneously allows the generation of a gene expression profile that is unique to the state of the cell. That is, normal tissue may be distinguished from a diseased tissue, and within a diseased tissue, different prognosis states (good or poor long term survival prospects, for example) may be determined. By comparing expression profiles of a diseased tissue in different states, information regarding which genes are important (including both up- and down-regulation of genes) in each of these states is obtained. The identification of sequences that are differentially expressed in a diseased tissue or normal tissue, as well as differential expression resulting in different prognostic outcomes, allows the use of this information in a number of ways. For example, a particular treatment regime may be evaluated. Similarly, diagnosis may be done or confirmed by comparing patient samples with the known expression profiles. Furthermore, these gene expression profiles (or individual genes) allow screening of drug candidates that suppress the expression of one or more pulmonory hypertension associated genes or convert a poor prognosis profile to a better prognosis profile.

The microarray may be fabricated using techniques known in the art. For example, probe oligonucleotides of an appropriate length are 5′-amine modified at position C6 and printed using commercially available microarray systems, e.g., the GENEMACHINE, OMNIGRID 100 MICROARRAYER and AMERSHAM CODELINK activated slides. Labeled cDNA oligomer corresponding to the target RNAs is prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g., 6×SSPE/30% formamide at 25° C. for 18 hours, followed by washing in 0.75×TNT at 37° C. for 40 minutes. At positions on the array where the immobilized probe DNA recognizes a complementary target cDNA in the sample, hybridization occurs. The labeled target cDNA marks the exact position on the array where binding occurs, allowing automatic detection and quantification. The output consists of a list of hybridization events, indicating the relative abundance of specific cDNA sequences, and therefore the relative abundance of the corresponding complementary mRNA, in the patient sample. According to one embodiment, the labeled cDNA oligomer is a biotin-labeled cDNA, prepared from a biotin-labeled primer. The microarray is then processed by direct detection of the biotin-containing transcripts using, e.g., STREPTAVIDIN-ALEXA647 conjugate, and scanned utilizing conventional scanning methods. Image intensities of each spot on the array are proportional to the abundance of the corresponding mRNA in the patient sample.

Other techniques for measuring mRNA gene expression are also within the skill in the art, and include various techniques for measuring rates of RNA transcription and degradation.

In one embodiment, provided herein is a method of diagnosing pulmonary hypertension in a subject, comprising the steps of obtaining a biological sample from the subject; analyzing the level of one or more markers in the biological sample; and comparing the expression of the one or more markers to a standard, for example, a pre-determined expression level of the correponding marker in a control sample. In one embodiment, if level of the one or more markers of test sample is different from the predetermined threshold unit, for example, more than 1.0, the subject is afflicted with pulmonary hypertension. The predetermined threshold unit may be any unit determined by one of skill in the art. For example, the predetermined threshold unit may be a ratio between a control sample value and a test sample value. The predetermined threshold unit may be 0.1, 0.2, 0.5, 1, 2, 5, 10, 50, 100, and 1000. In some embodiments, if the predetermined threshold has a range of values. In another embodiment, the expression level of one or more markers in test sample is greater than the predetermined threshold unit, for example greater than 1.0, then the subject has increased chance of being afflicted with pulmonary hypertension. In another embodiment, if the level of the one or more markers in test sample is greater than the predetermined threshold unit, for example greater than 1.0, then the subject has reduced chance of being afflicted with pulmonary hypertension pulmonary hypertension. In another embodiment, if the expression level of the one or more markers in test sample is less than the predetermined threshold unit, for example less than 1.0, then the subject is not afflicted with pulmonary hypertension. Increased chance or decreased chance may be measured by a statistical method well known to one of skill in the art.

In another embodiment, pulmonary hypertension is pulmonary arterial hypertension (PAH). In another embodiment, pulmonary hypertension is idiopathic pulmonary arterial hypertension. In another embodiment, signs of pulmonary hypertension include, but are not limited to, an increase in blood pressure in the pulmonary artery, pulmonary vein, or pulmonary capillaries. In another embodiment, symptoms of pulmonary hypertension include, but are not limited to, shortness of breath, dizziness, fainting, or any combination thereof. Other signs and symptoms are well known to one of skilled in the art.

In another embodiment, patients suffering from left heart failure are diagnosed by the methods of the invention. In another embodiment, patients suffering from systolic or diastolic malfunction of the left ventricle are diagnosed by the methods of the invention. In another embodiment, patients suffering from valvular dysfunction such as mitral regurgitation, mitral stenosis, aortic stenosis, or aortic regurgitation are diagnosed by the methods of the invention. In another embodiment, patients suffering from pulmonary edema or pleural effusions are diagnosed by the methods of the invention. In another embodiment, patients afflicted with HIV are diagnosed by the methods of the invention. In another embodiment, patients afflicted with scleroderma are diagnosed by the methods of the invention. In another embodiment, patients afflicted with congenital heart disease are diagnosed by the methods of the invention. In another embodiment, patients afflicted with an autoimmune disorder are diagnosed by the methods of the invention. In another embodiment, patients afflicted with a congenital heart disease are diagnosed by the methods of the invention. In another embodiment, patients afflicted with sickle cell disease are diagnosed by the methods of the invention. In another embodiment, patients afflicted with Human herpesvirus 8 are diagnosed by the methods of the invention. In another embodiment, patients afflicted with cirrhosis or other liver disease are diagnosed by the methods of the invention. In another embodiment, patients afflicted with portal hypertension are diagnosed by the methods of the invention. In another embodiment, patients afflicted with Kaposi's sarcoma are diagnosed by the methods of the invention. In another embodiment, patients afflicted with lung diseases are diagnosed by the methods of the invention. In another embodiment, patients afflicted with interstitial lung disease such as idiopathic pulmonary fibrosis, Pickwickian syndrome or obesity-hypoventilation syndrome are diagnosed by the methods of the invention. In another embodiment, patients afflicted with sleep apnea are diagnosed by the methods of the invention. In another embodiment, patients afflicted with sarcoidosis are diagnosed by the methods of the invention. In another embodiment, patients afflicted with histiocytosis are diagnosed by the methods of the invention. In another embodiment, patients afflicted with fibrosing mediastinitis are diagnosed by the methods of the invention. In another embodiment, patients afflicted with a thyroid disease are diagnosed by the methods of the invention. In another embodiment, patients afflicted with schistosomiasis are diagnosed by the methods of the invention. In another embodiment, patients afflicted with thromboembolism are diagnosed by the methods of the invention.

In another embodiment, a subject afflicted with any condition or disease that increases the chance of developing PH is diagnosed by the methods of the invention. In another embodiment, a subject having a family history of PH is diagnosed by the methods of the invention. In another embodiment, the disease is referred to as familial pulmonary arterial hypertension (FPAH). In another embodiment, the disease is referred to as idiopathic pulmonary arterial hypertension (IPAH).

In another embodiment, patients treated with the drug fenfluramine are diagnosed by the methods of the invention. In another embodiment, patients treated with the drug methamphetamine are diagnosed by the methods of the invention. In another embodiment, patients treated with the drug phentermine are diagnosed by the methods of the invention. In another embodiment, patients treated with the drug dexfenfluramine are diagnosed by the methods of the invention. In another embodiment, the disease is referred to as pulmonary veno-oclusive disease. In another embodiment, the disease is referred to as pulmonary capillary hemangiomatosis. In another embodiment, the disease is referred to as porto-pulmonary hypertension and associated with the presence of liver disease.

In another embodiment, the methods of the present invention are further combined with other methods that assess the condition of a subject suspected of being afflicted with PH. In another embodiment, the methods of the present invention are further combined with physical examination directed at typical signs of PH such as but not limited to: heart sounds, such as a widely split S2 or second heart sound, a loud P2 or pulmonic valve closure sound (part of the second heart sound), (para)sternal heave, possible S3 or third heart sound, and pulmonary regurgitation, elevated jugular venous pressure, peripheral edema (swelling of the ankles and feet), ascites (abdominal swelling due to the accumulation of fluid), hepatojugular reflux, clubbing, or any combination thereof.

In another embodiment, the methods of the present invention are further combined with HIV test. In another embodiment, the methods of the present invention are further combined with autoimmune disease tests. In another embodiment, the methods of the present invention are further combined with liver disease tests. In another embodiment, the methods of the present invention are further combined with electrocardiography (ECG). In another embodiment, the methods of the present invention are further combined with arterial blood gas measurements. In another embodiment, the methods of the present invention are further combined with X-rays of the chest (followed by high-resolution CT scanning if interstitial lung disease is suspected), and ventilation-perfusion or V/Q scanning to exclude or include chronic thromboembolic pulmonary hypertension. In another embodiment, the methods of the present invention are further combined with biopsy of the lung (interstitial lung disease). In another embodiment, the methods of the present invention are further combined with a “six-minute walk test”, i.e. the distance a patient can walk in six minutes. In another embodiment, the methods provided herein are further combined with the findings of an echocardiogram. In another embodiment, the methods provided herein are further combined with blood values of brain natriuretic protein (BNP) or pro-BNP. In another embodiment, the methods provided herein are further combined with blood values of uric acid. In another embodiment, the methods provided herein are further combined with blood values of troponin-T or other troponins.

In another embodiment, the methods provided herein, further comprise tests for Pulmonary artery occlusion pressure (PAOP or PCWP). In another embodiment, the methods provided herein, further comprise tests for pulmonary vascular resistance (PVR).

In another embodiment, the methods provided herein, are used for the prognosis of patients in WHO Group I—Pulmonary arterial hypertension (PAH). In another embodiment, the methods provided herein, are used for the prognosis of patients in WHO Group II—Pulmonary hypertension associated with left heart disease. In another embodiment, the methods provided herein, are used for the prognosis of patients in WHO Group III—Pulmonary hypertension associated with lung diseases and/or hypoxemia. In another embodiment, the methods provided herein, are used for the prognosis of patients in WHO Group IV—Pulmonary hypertension due to chronic thrombotic and/or embolic disease. In another embodiment, the methods provided herein, are used for the prognosis of patients in WHO Group V.

In another embodiment, patients diagnosed by the methods of the invention are further monitored. In another embodiment, monitoring comprises pulse oximetry. In another embodiment, monitoring comprises arterial blood gas tests. In another embodiment, monitoring comprises chest X-rays. In another embodiment, monitoring comprises serial ECG tests. In another embodiment, monitoring comprises serial echocardiography. In another embodiment, monitoring comprises spirometry or more advanced lung function studies.

In one embodiment, the standard is determined by the expression level profile in a healthy subject or pool of subjects. In another embodiment, the standard is the average expression level profile taken from a pool of subjects. In another embodiment, the standard is the average expression level of at least two markers of the invention taken from a pool of subjects. In another embodiment, the standard is the mean expression level profile taken from a pool of subjects. In another embodiment, the standard is the mean expression level of at least two markers of the invention taken from a pool of subjects. In another embodiment, the standard is the median expression profile level taken from a pool of subjects. In another embodiment, the standard is the median expression level of at least two markers of the invention taken from a pool of subjects.

In another embodiment, the standard is the expression level profile in a subject or pool of subject correctly diagnosed as having pulmonary hypertension or pulmonary atrial hypertension. In another embodiment, the pool of subjects is ethnically heterogenous. In another embodiment the pool of subjects is composed of whites, blacks, hispanics, asians or their combination. In another embodiment the pool of subjects is composed of any ethnic group. In another embodiment the pool of subjects is composed of any combination of various ethnic group.

In another embodiment, the terms marker and antigen are used interchangebly.

In another embodiment, the biological sample is blood, sera, plasma, saliva, sperm, urine, mucous, cerebral spinal fluid, or any combination thereof.

In another embodiment, an antigen level is measurable. In another embodiment, an antigen level is upregulated in the peripheral blood of a PAH In another embodiment, an antigen level is downregulated in the peripheral blood of PAH patients.

In another embodiment a marker expression is upregulated. In another embodiment a marker expression is upregulated in blood, sera, plasma, saliva, sperm, urine, mucous, cerebral spinal fluid, or any combination thereof. In another embodiment a marker expression is upregulated in subjects having IPAH when compared to the expression level of a healthy subject. In another embodiment a marker expression level is upregulated in subjects having IPAH when compared to the mean, the average, or the median expression level of a pool of healthy subjects. In another embodiment a marker expression level is upregulated in subjects having NIPAH when compared to the expression level of a healthy subject. In another embodiment a marker expression level is upregulated in subjects having NIPAH when compared to the mean, the average, or median expression level of a pool of healthy subjects. In another embodiment a marker expression level is upregulated in subjects having NIPAH when compared to the expression level of a subject with IPAH. In another embodiment a marker expression level is upregulated in subjects having NIPAH when compared to the mean, the average, or median expression level of a pool of subjects having IPAH. In another embodiment a marker expression level is downregulated. In another embodiment a marker expression level is downregulated in blood, sera, plasma, saliva, sperm, urine, mucous, cerebral spinal fluid, or their combination. In another embodiment a marker expression level is downregulated in subjects having IPAH when compared to the expression of a healthy subject. In another embodiment a marker expression level is downregulated in subjects having IPAH when compared to the the mean, the average, or median expression level of a pool of healthy subjects. In another embodiment a marker expression level is downregulated in subjects having NIPAH when compared to the expression of a healthy subject. In another embodiment a marker expression level is downregulated in subjects having NIPAH when compared to the mean, the average, or median expression level of a pool of healthy subjects. In another embodiment a marker expression level is downregulated in subjects having NIPAH when compared to the expression of a subject with IPAH. In another embodiment a marker expression level is downregulated in subjects having NIPAH when compared to the the mean, the average, or median expression level of a pool of subjects having IPAH.

In another embodiment a marker, taken from table 5 as provided herein below, is significantly upregulated in a subject afflicted with PAH, IPAH, NIPAH, or a combination thereof. In another embodiment the expression level of a marker, taken from table 5 as provided herein below, is significantly upregulated in a subject at risk of being afflicted with PAH, IPAH, NIPAH, or a combination thereof. In another embodiment, the expression level of one or more markers are significantly upregulated in a subject afflicted with PAH, IPAH, NIPAH, or a combination thereof. In another embodiment, the expression level of one or more markers are significantly upregulated in a subject at risk of being afflicted with PAH, IPAH, NIPAH, or a combination thereof. In another embodiment, expression level of one or more markers are significantly upregulated in a subject afflicted with PAH, IPAH, NIPAH, or a combination thereof. In another embodiment, expression level of one or more markers are significantly upregulated in a subject at risk of being afflicted with PAH, IPAH, NIPAH, or a combination thereof. In another embodiment expression level of 3 or more markers are significantly upregulated in a subject afflicted with PAH, IPAH, NIPAH, or a combination thereof. In another embodiment expression level of 3 or more markers are significantly upregulated in a subject at risk of being afflicted with PAH, IPAH, NIPAH, or a combination thereof. In another embodiment a marker expression level is significantly upregulated by about 1 to 2 fold in a subject afflicted with PAH, IPAH, NIPAH, or a combination thereof. In another embodiment a marker expression level is significantly upregulated by about 1 to 2 fold in a subject at risk of being afflicted with PAH, IPAH, NIPAH, or a combination thereof. In another embodiment a marker expression level is significantly upregulated by about 2.1 to 3 fold in a subject afflicted with PAH, IPAH, NIPAH, or a combination thereof. In another embodiment a marker expression level is significantly upregulated by about 2.1 to 3 fold in a subject at risk of being afflicted with PAH, IPAH, NIPAH, or a combination thereof. In another embodiment a marker expression level is significantly upregulated by about 3.1 to 4 fold in a subject afflicted with PAH, IPAH, NIPAH, or a combination thereof. In another embodiment a marker expression level is significantly upregulated by about 3.1 to 4 fold in a subject at risk of being afflicted with PAH, IPAH, NIPAH, or a combination thereof. In another embodiment a marker expression level is significantly upregulated by about 4.1 to 5 fold in a subject afflicted with PAH, IPAH, NIPAH, or a combination thereof. In another embodiment a marker expression level is significantly upregulated by about 4.1 to 5 fold in a subject at risk of being afflicted with PAH, IPAH, NIPAH, or a combination thereof. In another embodiment a marker expression level is significantly upregulated by about 5.1 to 6 fold in a subject afflicted with PAH, IPAH, NIPAH, or a combination thereof. In another embodiment a marker expression level is significantly upregulated by about 5.1 to 6 fold in a subject at risk of being afflicted with PAH, IPAH, NIPAH, or a combination thereof. In another embodiment a marker expression level is significantly upregulated by about 6.1 to 7 fold in a subject afflicted with PAH, IPAH, NIPAH, or a combination thereof. In another embodiment a marker expression level is significantly upregulated by about 6.1 to 7 fold in a subject at risk of being afflicted with PAH, IPAH, NIPAH, or a combination thereof. In another embodiment a marker expression level is significantly upregulated by about 7.1 to 8 fold in a subject afflicted with PAH, IPAH, NIPAH, or a combination thereof. In another embodiment a marker expression level is significantly upregulated by about 7.1 to 8 fold in a subject at risk of being afflicted with PAH, IPAH, NIPAH, or a combination thereof. In another embodiment a marker expression level is significantly upregulated by about 9.1 to 10 fold in a subject afflicted with PAH, IPAH, NIPAH, or a combination thereof. In another embodiment a marker expression level is significantly upregulated by about 9.1 to 10 fold in a subject at risk of being afflicted with PAH, IPAH, NIPAH, or a combination thereof. In another embodiment a marker expression level is significantly upregulated by about 11 to 20 fold in a subject afflicted with PAH, IPAH, NIPAH, or a combination thereof. In another embodiment a marker expression level is significantly upregulated by about 11 to 20 fold in a subject at risk of being afflicted with PAH, IPAH, NIPAH, or a combination thereof. In another embodiment a marker expression level is significantly upregulated by about 21 to 30 fold in a subject afflicted with PAH, IPAH, NIPAH, or a combination thereof. In another embodiment a marker expression level is significantly upregulated by about 21 to 30 fold in a subject at risk of being afflicted with PAH, IPAH, NIPAH, or a combination thereof. In another embodiment a marker expression level is significantly upregulated by about 31 to 40 fold in a subject afflicted with PAH, IPAH, NIPAH, or a combination thereof. In another embodiment a marker expression level is significantly upregulated by about 31 to 40 fold in a subject afflicted with PAH, IPAH, NIPAH, or a combination thereof. In another embodiment a marker expression level is significantly upregulated by about 31 to 40 fold in a subject at risk of being afflicted with PAH, IPAH, NIPAH, or a combination thereof. In another embodiment a marker expression level is significantly upregulated by about 41 to 50 fold in a subject afflicted with PAH, IPAH, NIPAH, or a combination thereof. In another embodiment a marker expression level is significantly upregulated by about 51 to 60 fold in a subject afflicted with PAH, IPAH, NIPAH, or a combination thereof. In another embodiment a marker expression level is significantly upregulated by about 51 to 60 fold in a subject at risk of being afflicted with PAH, IPAH, NIPAH, or a combination thereof. In another embodiment a marker expression level is significantly upregulated by about 61-70 fold in a subject afflicted with PAH, IPAH, NIPAH, or a combination thereof. In another embodiment a marker expression level is significantly upregulated by about 61-70 fold in a subject at risk of being afflicted with PAH, IPAH, NIPAH, or a combination thereof. In another embodiment a marker expression level is significantly upregulated by about 71 to 80 fold in a subject afflicted with PAH, IPAH, NIPAH, or a combination thereof. In another embodiment a marker expression level is significantly upregulated by about 71 to 80 fold in a subject at risk of being afflicted with PAH, IPAH, NIPAH, or a combination thereof. In another embodiment a marker expression level is significantly upregulated by 81 to 90 fold in a subject afflicted with PAH, IPAH, NIPAH, or a combination thereof. In another embodiment a marker expression level is significantly upregulated by 81 to 90 fold in a subject at risk of being afflicted with PAH, IPAH, NIPAH, or a combination thereof. In another embodiment a marker expression level is significantly upregulated by about 91 to 100 fold in a subject afflicted with PAH, IPAH, NIPAH, or a combination thereof. In another embodiment a marker expression level is significantly upregulated by about 91 to 100 fold in a subject at risk of being afflicted with PAH, IPAH, NIPAH, or a combination thereof. In another embodiment a marker expression level is significantly upregulated by about 101-120 fold in a subject afflicted with PAH, IPAH, NIPAH, or a combination thereof. In another embodiment a marker expression level is significantly upregulated by about 101-120 fold in a subject at risk of being afflicted with PAH, IPAH, NIPAH, or a combination thereof. In another embodiment a marker expression level is significantly upregulated by about 121 to 147 fold in a subject afflicted with PAH, IPAH, NIPAH, or a combination thereof. In another embodiment a marker expression level is significantly upregulated by about 121 to 147 fold in a subject at risk of being afflicted with PAH, IPAH, NIPAH, or a combination thereof.

In another embodiment the two markers that are significantly upregulated comprise Alpha-1 antitrypsin along with any one of the following markers: adiponectin, alpha-fetoprotein, Beta-2 microglobulin, cancer antigen 125, cancer antigen 19-9, CD40, C reactive protein, endothelin-1, EN-RAGE, eotaxin, erythropoietin, fatty acid binding protein, fibrinogen, G-CSF, growth hormone, GM-CSF, ICAM-1, IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1, FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise adiponectin along with any one of the following markers: alpha-fetoprotein, Beta-2 microglobulin, cancer antigen 125, cancer antigen 19-9, CD40, C reactive protein, endothelin-1, EN-RAGE, eotaxin, erythropoietin, fatty acid binding protein, fibrinogen, G-CSF, growth hormone, GM-CSF, ICAM-1, IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1, FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise alpha-fetoprotein along with any one of the following markers: Beta-2 microglobulin, cancer antigen 125, cancer antigen 19-9, CD40, C reactive protein, endothelin-1, EN-RAGE, eotaxin, erythropoietin, fatty acid binding protein, fibrinogen, G-CSF, growth hormone, GM-CSF, ICAM-1, IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1, FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise Beta-2 microglobulin along with any one of the following markers: cancer antigen 125, cancer antigen 19-9, CD40, C reactive protein, endothelin-1, EN-RAGE, eotaxin, erythropoietin, fatty acid binding protein, fibrinogen, G-CSF, growth hormone, GM-CSF, ICAM-1, IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1 beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise cancer antigen 125 along with any one of the following markers: cancer antigen 19-9, CD40, C reactive protein, endothelin-1, EN-RAGE, eotaxin, erythropoietin, fatty acid binding protein, fibrinogen, G-CSF, growth hormone, GM-CSF, ICAM-1, IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise cancer antigen 19-9along with any one of the following markers: CD40, C reactive protein, endothelin-1, EN-RAGE, eotaxin, erythropoietin, fatty acid binding protein, fibrinogen, G-CSF, growth hormone, GM-CSF, ICAM-1, IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise CD40 along with any one of the following markers: C reactive protein, endothelin-1, EN-RAGE, eotaxin, erythropoietin, fatty acid binding protein, fibrinogen, G-CSF, growth hormone, GM-CSF, ICAM-1, IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise a C reactive protein along with any one of the following markers: endothelin-1, EN-RAGE, eotaxin, erythropoietin, fatty acid binding protein, fibrinogen, G-CSF, growth hormone, GM-CSF, ICAM-1, IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise endothelin-1 along with any one of the following markers: EN-RAGE, eotaxin, erythropoietin, fatty acid binding protein, fibrinogen, G-CSF, growth hormone, GM-CSF, ICAM-1, IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1 alpha, MIP-1 beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise EN-RAGE along with any one of the following markers: eotaxin, erythropoietin, fatty acid binding protein, fibrinogen, G-CSF, growth hormone, GM-CSF, ICAM-1, IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise eotaxin along with any one of the following markers: erythropoietin, fatty acid binding protein, fibrinogen, G-CSF, growth hormone, GM-CSF, ICAM-1, IgA, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise erythropoietin along with any one of the following markers: fatty acid binding protein, fibrinogen, G-CSF, growth hormone, GM-CSF, ICAM-1, IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise fatty acid binding protein along with any one of the following markers: fibrinogen, G-CSF, growth hormone, GM-CSF, ICAM-1, IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise fibrinogen along with any one of the following markers: G-CSF, growth hormone, GM-CSF, ICAM-1, IgA, IL-10, IL-12p40, IL-12p70, IL-13, IL-15, IL-16 IL-1ra, IL-2, IL-3, IL-4, IL-5,

IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise G-CSF along with any one of the following markers: growth hormone, GM-CSF, ICAM-1, IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RH, TNF-alpha, TNF-beta, thyroid stimulating hormone, VCAM-1, VEGF, von Willebrand factor.

In another embodiment the two markers that are significantly upregulated comprise growth hormone along with any one of the following markers: GM-CSF, ICAM-1, IgA, IL-10, IL-12p40, IL-12p70, IL-13, IL-15, IL-16 IL-1 ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise GM-CSF along with any one of the following markers: ICAM-1, IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise ICAM-1 along with any one of the following markers: IgA, IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise IgA along with any one of the following markers: IL-10, IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise IL-10 along with any one of the following markers: IL-12p40, IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise IL-12p40 along with any one of the following markers: IL-12p70, IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise IL-12p70 along with any one of the following markers: IL-13, IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise IL-13 along with any one of the following markers: IL-15, IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise IL-15 along with any one of the following markers: IL-16 IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise IL-16 along with any one of the following markers: IL-1ra, IL-2, IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise IL-2 along with any one of the following markers: IL-3, IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise IL-3 along with any one of the following markers: IL-4, IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise IL-4 along with any one of the following markers: IL-5, IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise IL-5 along with any one of the following markers: IL-6, IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise IL-6 along with any one of the following markers: IL-8, insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RH, TNF-alpha, TNF-beta, thyroid stimulating hormone, VCAM-1, VEGF, von Willebrand factor.

In another embodiment the two markers that are significantly upregulated comprise IL-8 along with any one of the following markers: insulin, leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise insulin with any one of the following markers: leptin, MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise leptin with any one of the following markers: MCP-1, MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise MCP-1 with any one of the following markers: MDC, MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise MDC with any one of the following markers: MIP-1alpha, MIP-1beta, MMP-2, MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise MIP-1alpha with any one of the following markers: MMP-2, MMP-9, Myeloperoxidase, Myoglobin PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise MMP-2 with any one of the following markers: MMP-9, Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise MMP-9 with any one of the following markers: Myeloperoxidase, Myoglobin, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise Myeloperoxidase with any one of the following markers: Myoglobin, PAI-1, PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise Myoglobin with any one of the following markers: PAI-1 FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise PAI-1 with any one of the following markers: FGF basic, Prostatic acid phosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise FGF basic with any one of the following markers: Prostatic acidphosphatase, PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise Prostatic acid phosphatase with any one of the following markers: PAPP-A, serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise PAPP-A with any one of the following markers: serum amyloid P, Stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise serum amyloid P with any one of the following markers: stem cell factor, SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise stem cell factor with any one of the following markers: SHBG, thyroxine binding globulin, Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise thyroxine binding globulin with any one of the following markers: Tenascin C, Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise tenascin C binding globulin with any one of the following markers: Tissue factor, TIMP-1, TNF RII, TNF-alpha, TNF-beta, thyroid stimulating hormone, VCAM-1, VEGF, von Willebrand factor.

In another embodiment the two markers that are significantly upregulated comprise tissue factor binding globulin with any one of the following markers: TIMP-1, TNF RII, TNF-alpha, TNF-beta, thyroid stimulating hormone, VCAM-1, VEGF, von Willebrand factor.

In another embodiment the two markers that are significantly upregulated comprise TIMP-1 with any one of the following markers: TNF RII, TNF-alpha, TNF-beta, thyroid stimulating hormone, VCAM-1, VEGF, von Willebrand factor.

In another embodiment the two markers that are significantly upregulated comprise TNF RII with any one of the following markers: TNF-alpha, TNF-beta, thyroid stimulating hormone another embodiment the two markers that are significantly upregulated comprise TNF-alpha with any one of the following markers: TNF-beta, thyroid stimulating hormone, VCAM-1, VEGF, von Willebrand factor.

In another embodiment the two markers that are significantly upregulated comprise TNF-beta with any one of the following markers: thyroid stimulating hormone, VCAM-1, VEGF, von Willebrand factor.

In another embodiment the two markers that are significantly upregulated comprise thyroid stimulating hormone with any one of the following markers: VCAM-1, VEGF, von Willebrand factor.

In another embodiment the two markers that are significantly upregulated comprise VCAM-1 with any one of the following markers: VEGF, von Willebrand factor.

In another embodiment the two markers that are significantly upregulated VEGF and von Willebrand factor, VCAM-1, VEGF, von Willebrand factor.

In another embodiment a marker, taken from table 5 as provided herein below, is significantly downregulated in a subject afflicted with PAH, IPAH, NIPAH, or a combination thereof. In another embodiment a marker, taken from table 5 as provided herein below, is significantly downregulated in a subject at risk of being afflicted with PAH, IPAH, NIPAH, or a combination thereof. In another embodiment, one or more markers are significantly downregulated in a subject afflicted with PAH, IPAH, NIPAH, or a combination thereof. In another embodiment, one or more markers are significantly downregulated in a subject at risk of being afflicted with PAH, IPAH, NIPAH, or a combination thereof. In another embodiment, one or more markers are significantly downregulated in a subject afflicted with PAH, IPAH, NIPAH, or a combination thereof. In another embodiment, one or more markers are significantly downregulated in a subject at risk of being afflicted with PAH, IPAH, NIPAH, or a combination thereof. In another embodiment 3 or more markers are significantly downregulated in a subject afflicted with PAH, IPAH, NIPAH, or a combination thereof. In another embodiment 3 or more markers are significantly downregulated in a subject at risk of being afflicted with PAH, IPAH, NIPAH, or a combination thereof. In another embodiment a marker expression level is signficantly downregulated in a subject afflicted with PAH, IPAH, NIPAH, or a combination thereof. In another embodiment a marker expression level is signficantly downregulated in a subject at risk of being afflicted with PAH, IPAH, NIPAH, or a combination thereof. In another embodiment, a marker expression level is downregulated. In another embodiment, a marker expression level is significantly downregulated, in a subject afflicted with PAH, IPAH, NIPAH, or a combination thereof. In another embodiment, a marker expression level is significantly downregulated, in a subject at risk of being afflicted with PAH, IPAH, NIPAH, or a combination thereof. In another embodiment a marker expression level is significantly downregulated by 0.1 to 0.5 fold in a subject afflicted with PAH, IPAH, NIPAH, or a combination thereof. In another embodiment a marker expression level is significantly downregulated by 0.1 to 0.5 fold in a subject at risk afflicted with PAH, IPAH, NIPAH, or a combination thereof. In another embodiment the marker's expression is significantly downregulated by 0.6 to 1.0 fold in a subject afflicted with PAH, IPAH, NIPAH, or a combination thereof. In another embodiment the marker's expression is significantly downregulated by 0.6 to 1.0 fold in a subject at risk of being afflicted with PAH, IPAH, NIPAH, or a combination thereof. In another embodiment the marker's expression is significantly downregulated by 1.1 to 1.7 fold in a subject afflicted with PAH, IPAH, NIPAH, or a combination thereof. In another embodiment the marker's expression is significantly downregulated by 1.1 to 1.7 fold in a subject at risk of being afflicted with PAH, IPAH, NIPAH, or a combination thereof.

In another embodiment two markers that are significantly upregulated comprise calcitonin along with any one of the following markers: factor VII, FGF basic, IGF-1, IL-13, IL-15, IL-1 alpha, Lymphotactin, Prostatic acid phosphatase, PAPP-A, Prostate specific antigen free, RANTES, Serum amyloid P, SGOT, TNF-alpha, von Willebrand factor.

In another embodiment a marker, taken from table 5 as provided herein below, is significantly downregulated in a subject afflicted with PAH, IPAH, NIPAH, or a combination thereof. In another embodiment a marker, taken from table 5 as provided herein below, is significantly downregulated in a subject at risk of being afflicted with PAH, IPAH, NIPAH, or a combination thereof. In another embodiment, one or more markers are significantly downregulated in a subject afflicted with PAH, IPAH, NIPAH, or a combination thereof. In another embodiment, one or more markers are significantly downregulated in a subject at risk of being afflicted with PAH, IPAH, NIPAH, or a combination thereof. In another embodiment, one or more markers are significantly downregulated in a subject afflicted with PAH, IPAH, NIPAH, or a combination thereof. In another embodiment, one or more markers are significantly downregulated in a subject at risk of being afflicted with PAH, IPAH, NIPAH, or a combination thereof. In another embodiment 3 or more markers are significantly downregulated in a subject afflicted with PAH, IPAH, NIPAH, or a combination thereof. In another embodiment 3 or more markers are significantly downregulated in a subject at risk of being afflicted with PAH, IPAH, NIPAH, or a combination thereof. In another embodiment a marker expression level is signficantly downregulated in a subject afflicted with PAH, IPAH, NIPAH, or a combination thereof. In another embodiment a marker expression level is signficantly downregulated in a subject at risk of being afflicted with PAH, IPAH, NIPAH, or a combination thereof. In another embodiment, a marker expression level is downregulated. In another embodiment, a marker expression level is significantly downregulated, in a subject afflicted with PAH, IPAH, NIPAH, or a combination thereof. In another embodiment, a marker expression level is significantly downregulated, in a subject at risk of being afflicted with PAH, IPAH, NIPAH, or a combination thereof. In another embodiment a marker expression level is significantly downregulated by 0.1 to 0.5 fold in a subject afflicted with PAH, IPAH, NIPAH, or a combination thereof. In another embodiment a marker expression level is significantly downregulated by 0.1 to 0.5 fold in a subject at risk afflicted with PAH, IPAH, NIPAH, or a combination thereof. In another embodiment the marker's expression is significantly downregulated by 0.6 to 1.0 fold in a subject afflicted with PAH, IPAH, NIPAH, or a combination thereof. In another embodiment the marker's expression is significantly downregulated by 0.6 to 1.0 fold in a subject at risk of being afflicted with PAH, IPAH, NIPAH, or a combination thereof. In another embodiment the marker's expression is significantly downregulated by 1.1 to 1.7 fold in a subject afflicted with PAH, IPAH, NIPAH, or a combination thereof. In another embodiment the marker's expression is significantly downregulated by 1.1 to 1.7 fold in a subject at risk of being afflicted with PAH, IPAH, NIPAH, or a combination thereof.

In another embodiment two markers that are significantly upregulated comprise calcitonin along with any one of the following markers: factor VII, FGF basic, IGF-1, IL-13, IL-15, IL-1 alpha, Lymphotactin, Prostatic acid phosphatase, PAPP-A, Prostate specific antigen free, RANTES, Serum amyloid P, SGOT, TNF-alpha, von Willebrand factor.

In another embodiment two markers that are significantly upregulated comprise factor VII along with any one of the following markers: FGF basic, IGF-1, IL-13, IL-15, IL-1alpha, Lymphotactin, Prostatic acid phosphatase, PAPP-A, Prostate specific antigen free, RANTES, Serum amyloid P, SGOT, TNF-alpha, von Willebrand factor.

In another embodiment two markers that are significantly upregulated comprise FGF basic along with any one of the following markers:, IGF-1, IL-13, IL-15, IL-1 alpha, Lymphotactin, Prostatic acid phosphatase, PAPP-A, Prostate specific antigen free, RANTES, Serum amyloid P, SGOT, TNF-alpha, von Willebrand factor.

In another embodiment two markers that are significantly upregulated comprise IGF-1 along with any one of the following markers: IL-13, IL-15, IL-1alpha, Lymphotactin, Prostatic acid phosphatase, PAPP-A, Prostate specific antigen free, RANTES, Serum amyloid P, SGOT, TNF-alpha, von Willebrand factor.

In another embodiment two markers that are significantly upregulated comprise IL-13 along with any one of the following markers: IL-15, IL-1alpha, Lymphotactin, Prostatic acid phosphatase, PAPP-A, Prostate specific antigen free, RANTES, Serum amyloid P, SGOT, TNF-alpha, von Willebrand factor.

In another embodiment two markers that are significantly upregulated comprise IL-15 along with any one of the following markers: IL-1alpha, Lymphotactin, Prostatic acid phosphatase, PAPP-A, Prostate specific antigen free, RANTES, Serum amyloid P, SGOT, TNF-alpha, von Willebrand factor.

In another embodiment two markers that are significantly upregulated comprise IL-1 alpha along with any one of the following markers: Lymphotactin, Prostatic acid phosphatase, PAPP-A, Prostate specific antigen free, RANTES, Serum amyloid P, SGOT, TNF-alpha, von Willebrand factor.

In another embodiment two markers that are significantly upregulated comprise Lymphotactin along with any one of the following markers: Prostatic acid phosphatase, PAPP-A, Prostate specific antigen free, RANTES, Serum amyloid P, SGOT, TNF-alpha, von Willebrand factor.

In another embodiment two markers that are significantly upregulated comprise Prostatic acid phosphatase along with any one of the following markers: PAPP-A, Prostate specific antigen free, RANTES, Serum amyloid P, SGOT, TNF-alpha, von Willebrand factor.

In another embodiment two markers that are significantly upregulated comprise PAPP-A basic along with any one of the following markers: Prostate specific antigen free, RANTES, Serum amyloid P, SGOT, TNF-alpha, von Willebrand factor.

In another embodiment two markers that are significantly upregulated comprise Prostate specific antigen free along with any one of the following markers:, RANTES, Serum amyloid P, SGOT, TNF-alpha, von Willebrand factor.

In another embodiment two markers that are significantly upregulated comprise RANTES along with any one of the following markers: Serum amyloid P, SGOT, TNF-alpha, von Willebrand factor.

In another embodiment two markers that are significantly upregulated comprise Serum amyloid P along with any one of the following markers: SOOT, TNF-alpha, von Willebrand factor.

In another embodiment two markers that are significantly upregulated comprise SGOT along with any one of the following markers: TNF-alpha, von Willebrand factor.

In another embodiment two markers that are significantly upregulated comprise TNF-alpha and von Willebrand factor.

In another embodiment, the one or more markers are any appropriate permutation of the markers of Table 5 hereinbelow. In another embodiment, three or more markers are any appropriate permutation of the markers of Table 5. In another embodiment, four or more markers are any appropriate permutation of the markers of Table 5. In another embodiment, five or more markers are any appropriate permutation of the markers of Table 5.

In another embodiment, molecules known to promote tumorigenesis are also present at higher levels in PAH patients. In another embodiment, a cancer-related factor and/or an angiogenic factor, such as but not limited to MMP-2, MMP-9, TN-C, FGF-2, VEGF, adiponectin, growth hormone, fibrinogen, PAI-1 or any combination thereof are upregulated in the plasma of PAH patients and are used as markers of the invention.

In one embodiment, provided herein is a method of determining the prognosis of a subject developing pulmonary hypertension comprising the steps of obtaining a biological sample from the subject; analyzing the level of one or more markers in the biological sample; and comparing the expression of the one or more markers to a standard. In another embodiment, if the standard is extrapulated as discussed hereinabove from a healthy subject or a pool of subjects and the level of the one or more markers is different than the standard set by a predetermined threshold of greater than 1.0, the subject has the risk of having pulmonary hypertension In another embodiment, if the standard is taken from a subject or pool of subjects correctly diagnosed with pulmonary hypertension and the level of the one or more markers is different than the standard set by a predetermined threshold of greater than 1.0, the subject is not afflicted with pulmonary hypertension. In another embodiment, the standard is taken from the average of the pool of subjects. In another embodiment, the standard is taken from the mean of the pool of subjects. In another embodiment, the standard is taken from the median of the pool of subjects.

In one embodiment, the term “threshold” refers to a cutoff point or level of a marker (e.g., Calcitonin) expression that has been established based on experimental or clinical data. In another embodiment, “threshold” is marker specific and although one marker may exceed the predetermined threshold level, other marker or markers may not. In some embodiments, “threshold level” refers to the amount of a gene product (e.g., mRNA or protein) in a sample or a calculated numerical value based on multiple variables. The threshold level is a cutoff point above or below which certain characteristics or outcomes (e.g., PAH, or IPAH) are likely to occur. Generally, values above or below a defined threshold level correlate with some other characteristic or outcome, such as increased risk of developing symptoms associated with the pathology. Example 2 describes the establishment of threshold levels of marker expression in PAH that correlate with detection and prognosis. In one embodiment, threshold levels can be determined using any suitable method (e.g., statistical analysis of a group of samples with a known outcome). In some embodiments, threshold values are used by clinicians in the diagnosis and characterization of IPAH in a subject.

In another embodiment, the methods provided herein, comprise protein level (amount) measurements. In another embodiment, the methods provided herein, comprise RNA measurements. In another embodiment, the methods provided herein, comprise mRNA measurements. In another embodiment, methods of measuring the expression level of a given protein used as a marker are known to one of average skill in the art. In another embodiment, methods of measuring the transcription level of a given RNA molecule encoding a protein used as a marker are known to one of average skill in the art. In another embodiment, methods of measuring the transcription level of a given mRNA molecule encoding a protein used as a marker are known to one of average skill in the art.

In another embodiment, provided herein is a kit for diagnosing or providing prognosis for a subject developing pulmonary hypertension, comprising equipment including, but not limited to, assays and analytical tools for the assays, both as described hereinbelow in the exemplification, reagents, standards and instructions for analyzing the expression level of one or more markers in a biological sample of the subject.

In another embodiment, the methods as provided herein are directed to diagnosing pulmonary hypertension. In another embodiment, the methods are directed to determining the prognosis of the subject developing pulmonary hypertension. In yet another embodiment, the methods are directed to diagnosing and/or determining the prognosis of a subject the developing pulmonary hypertension.

In another embodiment, “pulmonary hypertension” or “PH” as described herein, refers to any one or all of the symptoms associated with pulmonary hypertension. In another embodiment, the diagnostic categories of PH comprise of, PAH. In one embodiment the diagnostic categories of PH comprise of Primary Pulmonary Hypertension. The diagnostic categories of PH comprise of PAH that are related to other causes. In another embodiment, the diagnostic categories of PH comprise of pulmonary hypertension that is associated with disorders of the respiratory system. In one embodiment, the diagnostic categories of PH comprise of pulmonary hypertension due to chronic thrombotic or embolic disease. In another embodiment, the diagnostic categories of PH comprise of pulmonary hypertension due to disorders directly affecting the pulmonary blood vessels. In one embodiment, the diagnostic categories of PH comprise of PAH related to collagen vascular diseases. In another embodiment, the diagnostic categories of PH comprise of pulmonary hypertension associated with chronic HIV infection. In one embodiment, the diagnostic categories of PH comprise of pulmonary hypertension associated with drugs or toxins. The diagnostic categories of PH comprise of pulmonary venous hypertension in other discrete embodiments provided in the methods for diagnosis and prognosis described herein. In one embodiment, a PH patient may be at risk or currently suffering from heart failure due to the increase in central venous pressure due to, in another embodiment, manifestations of PH.

In one embodiment, both primary pulmonary hypertension (PPH), pulmonary arterial hypertension (SPAH), or both are diagnosed using the methods described herein, using different marker combination to differentiate primary from secondary pulmonary arterial hypertension (SPAH). In one embodiment, Primary pulmonary hypertension (PPH) is a syndrome of dyspnoea, fatigue, chest pain and syncope resulting from an increase in pulmonary vascular resistance leading to right ventricular failure and death. Conversely, Secondary pulmonary arterial hypertension (SPAH) is an adverse outcome of a variety of systemic disorders. These include collagen vascular diseases, chronic thromboembolism, human immunodeficiency virus, portopulmonary hypertension, and other diseases. Progression of SPAH may persist in certain embodiments despite stabilization of the underlying disease, thereby contributing to the poor quality of life and unfavorable survival in these patients. In one embodiment, endothelin receptor blockade therapy was effective in SPAH.

In another embodiment, the invention provides a kit for diagnosis of pulmonary hypertension, comprising: antibodies that bind to one or more markers set forth in Table 5; and optionally, reagents for the formation of the medium favorable to the immunological reaction. In another embodiment, the invention provides a kit for diagnosis of pulmonary hypertension, comprising: oligonucleotides complementary to one or more markers set forth in Table 5; and optionally, reagents for the hybridization between said oligonucleotides and said markers. In another embodiment, the invention provides a kit to monitor a response to a therapy for pulmonary hypertension, comprising: antibodies that bind to one or more markers set forth in Table 5; and optionally, reagents for the formation of the medium favorable to the immunological reaction. In another embodiment, the invention provides a kit to monitor a response to a therapy for pulmonary hypertension, comprising: oligonucleotides complementary to one or more markers set forth in Table 5; and optionally, reagents for the hybridization between said oligonucleotides and said markers.

In some embodiments, a kit of the invention optionally includes directions for monitoring the levels of a protein or nucleic acid marker in a biological sample derived from a subject. In another embodiment, the kit comprises a sterile container which contains the primer, probe, or other detection regents; such containers can be boxes, ampoules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the antibodies, primers or probes described herein and their use in diagnosing pulmonary hypertension. Preferably, the kit further comprises any one or more of the reagents described in the diagnostic assays described herein. In other embodiments, the instructions include at least one of the following: description of the antibody, primer or probe; methods for using the enclosed materials for the diagnosis of pulmonary hypertension; precautions; warnings; indications; clinical or research studies; and/or references. The instructions may be printed directly on the container (when present), or as a label applied to the container, or as a separate sheet, pamphlet, card, or folder supplied in or with the container.

In one embodiment, the methods described herein are effective in the diagnosis or prognosis or monitoring of borderline pulmonary hypertension. In another embodiment, borderline PAH refers to those incidents where pulmonary intravenous pressure is between about 31 and 40 mm Hg.

In another embodiment, the invention provides a method for predicting the progression of pulmonary hypertension, in a subject, comprising the steps of: obtaining a biological sample from said subject; and testing said biological sample to determine whether or not a plurality of markers is over-expressed or under-expressed in said biological sample, relative to the expression of said plurality of markers in a control sample, whereby the over-expression or under-expression of said plurality of markers in said biological sample indicates the progression of said pulmonary hypertension, and whereby said plurality of markers comprises one or more markers set forth in Table 5.

In another embodiment, the invention provides a method of providing prognosis for pulmonary hypertension, in a subject, comprising the steps of: obtaining a biological sample from said subject; and testing said biological sample to determine whether or not a plurality of markers is over-expressed or under-expressed in said biological sample, relative to the expression of said plurality of markers in a control sample, whereby the over-expression or under-expression of said plurality of markers in said biological sample indicates said pulmonary hypertension, and whereby said plurality of markers comprises one or more markers set forth in Table 5.

In another embodiment, the invention provides a method of treating pulmonary hypertension, in a subject, comprising the steps of: obtaining a biological sample from said subject; and testing said biological sample to determine whether or not a plurality of markers is over-expressed or under-expressed in said biological sample, relative to the expression of said plurality of markers in a control sample, whereby the over-expression or under-expression of said plurality of markers in said biological sample indicates said pulmonary hypertension, and whereby said plurality of markers comprises one or more markers set forth in Table 5; and treating said pulmonary hypertension.

In another embodiment, the invention provides a method of screening a library of compounds to identify a compound to treat pulmonary hypertension, comprising the steps of: testing a biological sample to determine whether or not a plurality of markers is over-expressed or under-expressed in said biological sample, relative to the expression of said plurality of markers in a control sample, whereby said plurality of markers comprises one or more markers set forth in Table 5, thereby screening said library of compounds.

In one embodiment, endothelin expression is a marker described in example 2 as part of the markers encompassed by the methods provided herein.

The term “significant” as used herein indicates a statistically significant (p<0.05) value or values.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may comprise dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Materials and Methods

### Patients & Controls

Subjects with either IPAH or NIPAH were recruited from established and newly evaluated patients at the Pulmonary Vascular Disease Program at the University of Pennsylvania. PAH was defined according to standard diagnostic criteria, including a mean pulmonary artery pressure (PAP) greater than 25 mm Hg at rest or 30 mm Hg with exertion, and the absence of left atrial hypertension, significant hypoxemia, lung disease, or chronic venous thromboembolic disease. Consecutive patients aged 18 years or older were recruited during both outpatient and inpatient evaluations. This study was approved by the Institutional Review Board of the University of Pennsylvania, and blood samples were obtained following a patient's written informed consent. Blood was collected by venipuncture or from the proximal port of a pulmonary artery catheter already in place for hemodynamic testing as part of routine clinical care. No testing was performed or therapies provided as part of this study. Control plasma was obtained from healthy volunteers, as approved by the Institutional Review Board of the University of Pennsylvania. All subjects gave informed consent, and gave blood only on one occasion.

### Plasma Collection

Immediately following collection in potassium EDTA vacutainers (BD Biosciences), blood samples were stored upright for 30 min in collection tubes, before being centrifuged at RT for 15 minutes (min). at 3400 rpm. After centrifugation, the upper plasma layer, the intermediate buffy coat layer, and the bottom layer containing erythrocytes were separated using disposable plastic pipettes. Following fractionation, plasma was centrifuged, aliquoted, frozen and stored at −80° C.

### MAP Analysis for Plasma Antigens

200 μl of plasma from each patient was shipped on dry ice to Rules Based Medicine (RBM; Austin, Tex.). Thereafter, each sample was thawed at room temprature (RT), vortexed, spun at 13,000×g for 5 min. for clarification, and 40 μL was removed for MAP antigen analysis into a master microtiter plate. Using automated pipetting, an aliquot of each sample was introduced into one of the capture microsphere multiplexes of the Human Antigen MAP Version 1.6 (RBM). These mixtures of sample and capture microspheres were thoroughly mixed and incubated at RT for 1 hour. Multiplexed cocktails of biotinylated, reporter antibodies for each multiplex were then added robotically and after thorough mixing, were incubated for an additional hour at RT. Multiplexes were developed using an excess of streptavidin-phycoerythrin solution which was thoroughly mixed into each multiplex and incubated for 1 hour at RT. The volume of each multiplexed reaction was reduced by vacuum filtration and the volume increased by dilution into matrix buffer for analysis. Analysis was performed in a Luminex 100 instrument and the resulting data stream was interpreted using proprietary data analysis software developed at RBM and licensed to Qiagen Instruments and Upstate Biotechnology. For each multiplex, both calibrators and controls were included on each microtiter plate. 8-point calibrators were run in the first and last column of each plate and 3-level controls were included in duplicate. Test results were determined first for the high, medium and low controls for each multiplex to ensure proper assay performance. Unknown values for each of the analytes localized in a specific multiplex were determined using 4 and 5 parameter, weighted and non-weighted curve fitting algorithms included in the data analysis package. Eighty eight antigens were evaluated, including fetal antigens, cytokines, chemokines, proteases and ECM proteins using MAP Version 1.6. Quantitative data generated by MAPs were then mined for distinctive patterns of markers between IPAH and NIPAH patients and normal volunteers.

### ELISA for Tenascin-C

The levels of plasma-associated TN-C were determined using an ELISA kit with two monoclonal antibodies, 4F10TT and 19C4MS (IBL, Gunma, Japan). Measurements were conducted at 450 nm according to the manufacturer's instructions. Fold increases between controls and IPAH patients were then compared to results obtained via MAP analysis.

Data acquisition & Statistical Analyses

Clinical data were gathered and stored in MT-Health Software (TodoSoft Uruguay, Montevideo, Uruguay). Gathered information included the following: demographics, symptoms, history and diagnosis, treatments, physical exam results, clinical laboratory results, pulmonary function test results, echocardiogram data, cardiac catheterization and 6 minute walk test results. Only patients with complete demographic, New York Heart Association (NYHA) functional classification and catheterization data were included in the final analysis. After MAP analysis of plasma, data were analyzed using SigmaStat and SigmaPlot software (Version 3.5 & 10.0, Systat Software IncSan Jose, Calif.). Multiple groups of data were compared by Kruskal-Wallis 1-way analysis of variance (ANOVA), and if significant (p<0.05), they were further evaluated using Dunn's multiple comparison test. Data were presented as mean±standard deviation, median, mean, fold change, or frequency (percentage). Box Plots were used to graphically display the results. The authors had full access to the data and take responsibility for its integrity. All authors nave read and agree to the manuscript as written.

### RESULTS

### Example 1

### Patient Characteristics

Plasma from 51 healthy volunteers and 113 PAH patients was evaluated. Clinical and/or demographic data for patients and volunteers are displayed in Table I. Fifty eight patients had IPAH, whereas 55 had either FPAH (n=5) or APAH (n=50). For this study, the FPAH and APAH patients were evaluated together, and are designated as the non-IPAH (NIPAH) group. Of the APAH patients, 13 had portopulmonary hypertension, 6 had PAH associated with anorectic agent use and 18 with collagen vascular disease (i.e. 3 with systemic lupus erythematosis and 15 with scleroderma-associated PAH). Thirteen patients had other associated forms of PAH, including 1 with hereditary hemorrhagic telangiestasia and 1 with glycogen storage disease. Of the remainder, 3 had pulmonary veno-occlusive disease (PVOD), 2 HIV infection, 2 had atrial septal defects (ASD), 1 ventricular septal defect (VSD), whereas 3 had other forms of congenital heart disease.

TABLE 1
Demographic & clinical characteristics.

| | IPAH | | NIPAH | | Control | |
|---|---|---|---|---|---|---|
| Variable | (n = 58) | | (n = 55) | | (n = 51) | |
| IPAH n (%) | 58 | (100%) | | | | |
| NIPAH n (%) | | | | | | |
| FPAH | | | 5 | (9%) | | |
| PPHTN | | | 13 | (24%) | | |
| Drugs & Toxins | | | 6 | (11%) | | |
| Collagen Vascular | | | 18 | (33%) | | |
| Disease | | | | | | |
| Other | | | 13 | (23%) | | |
| Age (years) | 51 ± 15 | | 50 ± 11 | | 28 ± 6 | |
| Gender, n (%) | | | | | | |
| Female | 45 | (77%) | 40 | (72%) | 25 | (49%) |
| Male | 13 | (23%) | 15 | (28%) | 26 | (51%) |
| Race/Ethnicity, n (%) | | | | | | |
| White (not of | 47 | (81%) | 42 | (76%) | 40 | (78%) |
| Hispanic origin) | | | | | | |
| Black (not of | 6 | (10%) | 7 | (13%) | 4 | (8%) |
| Hispanic origin) | | | | | | |
| Hispanic | 4 | (7%) | 5 | (9%) | 1 | (2%) |
| Asian | 1 | (2%) | 1 | (2%) | 6 | (12%) |
| NYHA/WHO func- | | | | | | |
| tional class, n (%) | | | | | | |
| I | 3 | (5%) | 0 | (0%) | | |
| II | 19 | (33%) | 16 | (29%) | | |
| III | 33 | (57%) | 37 | (67%) | | |
| IV | 3 | (5%) | 2 | (4%) | | |

| | | |
|---|---|---|
| Mean right atrial | 9.63 ± 7 | 9.77 ± 6.27 |
| pressure (mmHg) | | |
| Mean pulmonary | 47 ± 14 | 44.50 ± 13.28 |
| artery pressure | | |
| (mmHg) | | |
| Mean pulmonary | 10.91 ± 5.96 | 9.58 ± 4.49 |
| capillary wedge | | |
| pressure (mm Hg) | | |
| Pulmonary vascular | 789 ± 439 | 631.40 ± 2.65 |
| resistance | | |
| (dyn/s/cm⁻⁵) | | |
| Cardiac Index | 2.4 ± 0.89 | 2.65 ± 0.91 |
| (L/min/m²) | | |

Data are presented as mean +/− sd and frequency (percentage). IPAH: idiopathic pulmonary arterial hypertension,; NIPAH: non-idiopathic pulmonary arterial hypertension,; FPAH: familial pulmonary arterial hypertension, PPHTN: portopulmonary hypertension, NYHA: New York Heart Associatio, WHO: World Health Organization

### Example 2

### Map Analysis for Controls, Ipah and Nipah Patients

MAP analysis of PAH and healthy control plasma revealed significant differences in the levels of specific circulating antigens. As shown in Tables 2 and 3, numerous antigens were either significantly increased (in green) or decreased (in red) between groups. Thirty nine antigens were significantly (P<0.05) increased (n=35) or decreased (n=4) in all PAH patients when compared to healthy controls. Additionally, 10 antigens were uniquely elevated (n=8) or diminished (n=2) in IPAH patients when compared to controls, whereas 14 antigens were significantly increased (n=13) or decreased (n=1) solely in NIPAH patients versus controls. Importantly, significant differences in the levels of 22 antigens were noted between IPAH from NIPAH patients. Specific details about a number of these antigens are discussed below.

TABLE 2
MAP analysis data.

| | Control | Control | IPAH | Swiss-Prot |
|---|---|---|---|---|
| | vs | vs | vs | Accession |
| Antigen | IPAH | NIPAH | NIPAH | Number |
| Alpha-1 Antitrypsin | 1.23 | 1.43 | 1.17 | P07758 |
| Adiponectin | 2.67 | 2.71 | 1.01 | Q15848 |
| Alpha-2 Macroglobulin | 1.2 | 0.86 | 0.72 | P01023 |
| Alpha-Fetoprotein | 1.25 | 1.27 | 1.02 | P02771 |
| Apolipoprotein A1 | 0.9 | 1.08 | 1.21 | P02647 |
| Apolipoprotein CIII | 0.92 | 0.97 | 1.05 | P02656 |
| Apolipoprotein H | 0.91 | 0.97 | 1.06 | P02749 |
| Beta-2 Microglobulin | 2.7 | 3.05 | 1.13 | P23560 |
| Brain-Derived | 1.04 | 1.28 | 1.22 | P01884 |
| Neurotrophic Factor | | | | |
| Complement 3 | 1.01 | 1.05 | 1.04 | P01024 |
| Cancer Antigen 125 | 3.06 | 11.69 | 3.82 | Q14596 |
| Cancer Antigen 19-9 | 2.44 | 4.05 | 1.66 | Q9BXJ9 |
| Calcitonin | 0.44 | 0.71 | 1.58 | P01258 |
| CD40 | 2.54 | 2.74 | 1.07 | Q6P2H9 |
| CD40 Ligand | 1.12 | 1.44 | 1.28 | P29965 |
| Carcinoembryonic | 0.94 | 1.01 | 1.07 | P78448 |
| Antigen | | | | |
| Creatine Kinase-MB | 1.7 | 1.57 | 0.93 | P06732 |
| C Reactive Protein | 7.21 | 11.36 | 1.58 | P02741 |
| EGF | 1.44 | 1.02 | 0.71 | P01133 |
| ENA-78 | 0.82 | 1.04 | 1.27 | P42830 |
| Endothelin-1 | 8.56 | 4.23 | 0.49 | P05305 |
| EN-RAGE | 12.31 | 8.29 | 0.67 | P80511 |
| Eotaxin | 2.27 | 2.19 | 0.96 | P51671 |
| Erythropoietin | 85 | 146.89 | 1.72 | P01588 |
| Fatty Acid Binding | 2.77 | 2.87 | 1.03 | P05413 |
| Protein | | | | |
| Factor VII | 0.62 | 0.79 | 1.25 | P08709 |
| Ferritin | 1.13 | 1.82 | 1.61 | P02794 |
| FGF basic | 37.88 | 9.20 | 0.24 | P09038 |
| Fibrinogen | 1.64 | 1.50 | 0.91 | P02671 |
| G-CSF | 2.26 | 1.94 | 0.86 | P09919 |
| Growth Hormone | 1.01 | 1.73 | 1.71 | P01241 |
| GM-CSF | 2.23 | 1.80 | 0.81 | P04141 |
| Glutathione | 4.94 | 5.29 | 1.07 | P08263 |
| S-Transferase | | | | |
| Haptoglobin | 1.08 | 1.59 | 1.46 | P00738 |
| ICAM-1 | 2 | 2.64 | 1.35 | P05362 |
| IgA | 1.23 | 1.61 | 1.30 | AJ222547.1 * |
| IgE | 1.21 | 1.34 | 1.10 | AAD41764 * |
| IGF-1 | 0.3 | 0.60 | 1.64 | P01343 |
| IgM | 1.03 | 1.29 | 1.24 | BAA12061 * |
| IL-10 | 2.56 | 2.46 | 0.96 | P22301 |
| IL-12p40 | 6 | 3.79 | 0.64 | P29460 |
| IL-12p70 | 5.38 | 1.69 | 0.31 | P29460 |
| IL-13 | 1.12 | 0.81 | [symbol] | P35225 |
| IL-15 | 2 | 1.28 | 0.67 | P40993 |
| IL-16 | 5 | 4.13 | 0.84 | Q14005 |
| IL-18 | 2.65 | 2.59 | 0.97 | AJ295724.1 * |
| IL-1alpha | 0.11 | 0.01 | 0.09 | P18510 |
| IL-1beta | 16.84 | 7.55 | 0.45 | P01584 |
| IL-1ra | 5.7 | 12.43 | 2.17 | P18510 |
| IL-2 | 4.04 | 3.20 | 0.79 | P01585 |
| IL-3 | 3.18 | 2.53 | 0.79 | P08700 |
| IL-4 | 3.8 | 2.27 | 0.60 | P05112 |
| IL-5 | 2.03 | 1.86 | 0.91 | P05113 |
| IL-6 | 3.8 | 8.21 | 2.11 | P05231 |
| IL-7 | 0.99 | 0.98 | 0.99 | P13232 |
| IL-8 | 1.62 | 1.90 | 1.17 | P10145 |
| Insulin | 1.17 | 1.56 | 1.33 | P01308 |
| Leptin | 2.53 | 4.34 | 1.71 | P41159 |
| Lipoprotein (a) | 1.54 | 2.21 | 1.43 | P08519 |
| Lymphotactin | 0.73 | 0.52 | 0.70 | P47992 |
| MCP-1 | 3.73 | 3.42 | 0.92 | P13500 |
| MDC | 2 | 1.61 | 0.82 | O00626 |
| MIP-1alpha | 1 | 1.21 | 1.27 | P10147 |
| MIP-1beta | 2.43 | 3.68 | 1.51 | P13236 |
| MMP-2 | 1 | 1.69 | 1.67 | P08253 |
| MMP-3 | 0.01 | 0.04 | 3.27 | P08254 |
| MMP-9 | 1.57 | 2.49 | 1.58 | P14780 |
| Myeloperoxidase | 36.88 | 56.08 | 1.52 | P05164 |
| Myoglobin | 1.76 | 1.72 | 0.97 | P02144 |
| PAI-1 | 1.41 | 1.74 | 1.23 | P05121 |
| Prostatic Acid | 0.62 | 1.20 | 1.92 | P15309 |
| Phosphatase | | | | |
| PAPP-A | 3.98 | 1.75 | 0.44 | Q13219 |
| Prostate Specific | 0.23 | 0.40 | 1.65 | P07288 |
| Antigen, Free | | | | |
| RANTES | 1.1 | 1.30 | 1.18 | P13501 |
| Serum Amyloid P | 0.78 | 1.03 | 1.31 | P02743 |
| Stem Cell Factor | 0.96 | 1.48 | 1.53 | P21583 |
| SGOT | 0.46 | 0.80 | 1.71 | P17174 |
| SHBG | 1 | 1.15 | 1.14 | P04278 |
| Thyroxine | 1 | 1.18 | 1.19 | P05543 |
| Binding Globulin | | | | |
| Tenascin C | 1.14 | 2.06 | 1.80 | X78565.1 * |
| Tissue Factor | 0.8 | 1.98 | 2.46 | P13726 |
| TIMP-1 | 1.84 | 2.29 | 1.24 | P01033 |
| TNF RII | 2.27 | 2.74 | 1.20 | Q92956 |
| TNF-alpha | 18.8 | 11.02 | 0.59 | P01375 |
| TNF-beta | 2.6 | 2.36 | 0.91 | P01374 |
| Thrombopoietin | 1 | 0.93 | 0.94 | P40225 |
| Thyroid | 1.4 | 3.07 | 2.10 | P01215 |
| Stimulating Hormone | | | | |
| VCAM-1 | 2 | 2.37 | 1.22 | P19320 |
| VEGF | 1.04 | 2.55 | 2.43 | P15692 |
| von Willebrand | 2 | 1.86 | 0.95 | P04275 |
| Factor | | | | |

IPAH: idiopathic pulmonary hypertension, NIPAH: non-idiopathic pulmonary hypertension, red: significant decrease in antigen levels, green: significant increase in antigen levels.
* Embl protein access number.
* Embl protein accession number [symbol]
indicates data missing or illegible when filed

TABLE 3
Map Analysis Data: 51 Controls

| | Control | Control | IPAH |
|---|---|---|---|
| Antigen | vs IPAH | vs NIPAH | vs NIPAH |
| Alpha-1 Antitrypsin | 1.1 | 1.3 | 0.9 |
| Adiponectin | 2.4 | 2.4 | 1.0 |
| Alpha-2 Macroglobulin | 0.5 | 0.4 | 1.4 |
| Alpha-Fetoprotein | 1.1 | 1.2 | 1.0 |
| Apolipoprotein A1 | 0.8 | 1.0 | 0.8 |
| Apolipoprotein CIII | 0.7 | 0.8 | 1.0 |
| Apolipoprotein H | 0.9 | 1.0 | 0.9 |
| Beta-2 Microglobulin | 3.0 | 3.3 | 0.9 |
| Brain-Derived Neurotrophic Factor | 0.8 | 1.0 | 0.8 |
| Complement 3 | 1.1 | 1.1 | 1.0 |
| Cancer Antigen 125 | 3.6 | 13.6 | 0.3 |
| Cancer Antigen 19-9 | 1.2 | 2.0 | 0.6 |
| Calcitonin | 0.7 | 1.0 | 0.6 |
| CD40 | 2.0 | 2.2 | 0.9 |
| CD40 Ligand | 0.3 | 0.4 | 0.8 |
| Carcinoembryonic Antigen | 0.6 | 0.7 | 0.9 |
| Creatine Kinase-MB | 1.9 | 1.8 | 1.1 |
| C Reactive Protein | 7.6 | 14.5 | 0.5 |
| EGF | 0.7 | 0.5 | 1.4 |
| ENA-78 | 0.8 | 1.1 | 0.8 |
| Endothelin-1 | 2.8 | 1.4 | 2.0 |
| EN-RAGE | 16.2 | 11.9 | 1.4 |
| Eotaxin | 1.9 | 1.8 | 1.0 |
| Erythropoietin | 7.9 | 13.6 | 0.6 |
| Fatty Acid Binding Protein | 4.0 | 4.1 | 1.0 |
| Factor VII | 0.5 | 0.6 | 0.8 |
| Ferritin | 1.2 | 1.9 | 0.6 |
| FGF basic | 4.3 | 1.0 | 4.1 |
| Fibrinogen | 1.5 | 1.4 | 1.1 |
| G-CSF | 1.5 | 1.3 | 1.2 |
| Growth Hormone | 0.8 | 1.3 | 0.6 |
| GM-CSF | 3.1 | 2.5 | 1.2 |
| Glutathione S-Transferase | 1.3 | 1.4 | 0.9 |
| Haptoglobin | 1.0 | 1.5 | 0.7 |
| ICAM-1 | 1.6 | 2.2 | 0.7 |
| IgA | 1.3 | 1.7 | 0.8 |
| IgE | 1.7 | 1.8 | 0.9 |
| IGF-1 | 0.3 | 0.5 | 0.6 |
| IgM | 0.9 | 1.2 | 0.8 |
| IL-10 | 1.7 | 1.6 | 1.0 |
| IL-12p40 | 4.2 | 2.7 | 1.6 |
| IL-12p70 | 4.5 | 1.4 | 3.2 |
| IL-13 | 0.6 | 0.4 | 1.4 |
| IL-15 | 1.3 | 0.9 | 1.5 |
| IL-16 | 5.1 | 4.2 | 1.2 |
| IL-18 | 2.3 | 2.2 | 1.0 |
| IL-1alpha | 24.5 | 2.2 | 11.1 |
| IL-1beta | 4.7 | 2.1 | 2.2 |
| IL-1ra | 5.0 | 10.9 | 0.5 |
| IL-2 | 6.2 | 4.9 | 1.3 |
| IL-3 | 1.3 | 1.0 | 1.3 |
| IL-4 | 0.8 | 0.5 | 1.7 |
| IL-5 | 1.9 | 1.8 | 1.1 |
| IL-6 | 88.2 | 186.1 | 0.5 |
| IL-7 | 1.0 | 1.0 | 1.0 |
| IL-8 | 1.7 | 2.0 | 0.9 |
| Insulin | 1.3 | 1.7 | 0.8 |
| Leptin | 2.4 | 4.0 | 0.6 |
| Lipoprotein (a) | 1.6 | 2.3 | 0.7 |
| Lymphotactin | 1.0 | 0.7 | 1.4 |
| MCP-1 | 2.0 | 1.9 | 1.1 |
| MDC | 1.8 | 1.4 | 1.2 |
| MIP-1alpha | 0.8 | 1.1 | 0.8 |
| MlP-1beta | 2.1 | 3.2 | 0.7 |
| MMP-2 | 1.0 | 1.7 | 0.6 |
| MMP-3 | 1.6 | 5.3 | 0.3 |
| MMP-9 | 1.4 | 3.1 | 0.5 |
| Myeloperoxidase | 8.3 | 12.6 | 0.7 |
| Myoglobin | 2.0 | 2.0 | 1.0 |
| PAI-1 | 1.8 | 2.2 | 0.8 |
| Prostatic Acid Phosphatase | 0.7 | 1.4 | 0.5 |
| PAPP-A | 3.8 | 1.7 | 2.3 |
| Prostate Specific Antigen, Free | [symbol] | 0.5 | 0.6 |
| RANTES | 1.1 | 1.3 | 0.9 |
| Serum Amyloid P | 0.9 | 1.2 | 0.8 |
| Stem Cell Factor | 0.8 | 1.2 | 0.7 |
| SGOT | 0.6 | 1.0 | 0.6 |
| SHBG | 1.0 | 1.2 | 0.9 |
| Thyroxine Binding Globulin | 1.1 | 1.3 | 0.8 |
| Tenascin C | 1.0 | 1.8 | 0.6 |
| Tissue Factor | 1.1 | 2.6 | 0.4 |
| TIMP-1 | 1.8 | 2.2 | 0.8 |
| TNF RII | 2.2 | 2.6 | 0.8 |
| TNF-alpha | 4.4 | 2.6 | 1.7 |
| TNF-beta | 3.8 | 3.4 | 1.1 |
| Thrombopoietin | 1.0 | 0.9 | 1.1 |
| Thyroid Stimulating Hormone | 1.3 | 2.6 | 0.5 |
| VCAM-1 | 1.8 | 2.2 | 0.8 |
| VEGF | 0.8 | 2.0 | 0.4 |
| von Willebrand Factor | 2.4 | 2.3 | 1.1 |

[symbol]

indicates data missing or illegible when filed

TABLE 4
Summary of detected increases or decreases in markers.

| | SSD | SSD |
|---|---|---|
| Group comparison | increases | decreases |
| IPAH + NIPAH vs Control | 53 | 12 |
| IPAH vs Control | 6 | 5 |
| NIPAH vs Control | 11 | 1 |
| IPAH vs NIPAH | 8 | 14 |

IPAH: idiopathic pulmonary arterial hypertension; NIPAH: non-idiopathic pulmonary arterial hypertension; SSD: statical significant difference

TABLE 5
Markers for Palmonory Hypertension

| | Swiss-Prot |
|---|---|
| Antigen | Accession Number |
| Alpha-1 Antitrypsin | P07758 |
| Adiponectin | Q15848 |
| Alpha-2 Macroglobulin | P01023 |
| Alpha-Fetoprotein | P02771 |
| Apolipoprotein A1 | P02647 |
| Apolipoprotein CIII | P02656 |
| Apolipoprotein H | P02749 |
| Beta-2 Microglobulin | P23560 |
| Brain-Derived Neurotrophic Factor | P01884 |
| Complement 3 | P01024 |
| Cancer Antigen 125 | Q14596 |
| Cancer Antigen 19-9 | Q9BXJ9 |
| Calcitonin | P01258 |
| CD40 | Q6P2H9 |
| CD40 Ligand | P29965 |
| Carcinoembryonic Antigen | P78448 |
| Creatine Kinase-MB | P06732 |
| C Reactive Protein | P02741 |
| EGF | P01133 |
| ENA-78 | P42830 |
| Endothelin-1 | P05305 |
| EN-RAGE | P80511 |
| Eotaxin | P51671 |
| Erythropoietin | P01588 |
| Fatty Acid Binding Protein | P05413 |
| Factor VII | P08709 |
| Ferritin | P02794 |
| FGF basic | P09038 |
| Fibrinogen | P02671 |
| G-CSF | P09919 |
| Growth Hormone | P01241 |
| GM-CSF | P04141 |
| Glutathione S-Transferase | P08263 |
| Haptoglobin | P00738 |
| ICAM-1 | P05362 |
| IgA | AJ222547.1 * |
| IgE | AAD41764 * |
| IGF-1 | P01343 |
| IgM | BAA12061 * |
| IL-10 | P22301 |
| IL-12p40 | P29460 |
| IL-12p70 | P29460 |
| IL-13 | P35225 |
| IL-15 | P40993 |
| IL-16 | Q14005 |
| IL-18 | AJ295724.1 * |
| IL-1alpha | P18510 |
| IL-1beta | P01584 |
| IL-1ra | P18510 |
| IL-2 | P01585 |
| IL-3 | P08700 |
| IL-4 | P05112 |
| IL-5 | P05113 |
| IL-6 | P05231 |
| IL-7 | P13232 |
| IL-8 | P10145 |
| Insulin | P01308 |
| Leptin | P41159 |
| Lipoprotein (a) | P08519 |
| Lymphotactin | P47992 |
| MCP-1 | P13500 |
| MDC | O00626 |
| MIP-1alpha | P10147 |
| MIP-1beta | P13236 |
| MMP-2 | P08253 |
| MMP-3 | P08254 |
| MMP-9 | P14780 |
| Myeloperoxidase | P05164 |
| Myoglobin | P02144 |
| PAI-1 | P05121 |
| Prostatic Acid Phosphatase | P15309 |
| PAPP-A | Q13219 |
| Prostate Specific Antigen, Free | P07288 |
| RANTES | P13501 |
| Serum Amyloid P | P02743 |
| Stem Cell Factor | P21583 |
| SGOT | P17174 |
| SHBG | P04278 |
| Thyroxine Binding Globulin | P05543 |
| Tenascin C | X78565.1 * |
| Tissue Factor | P13726 |
| TIMP-1 | P01033 |
| TNF RII | Q92956 |
| TNF-alpha | P01375 |
| TNF-beta | P01374 |
| Thrombopoietin | P40225 |
| Thyroid Stimulating Hormone | P01215 |
| VCAM-1 | P19320 |
| VEGF | P15692 |
| von Willebrand Factor | P04275 |

* Embl protein accession number

### Example 3

### Comparing Map Analysis with Elisa for Tenascin-C

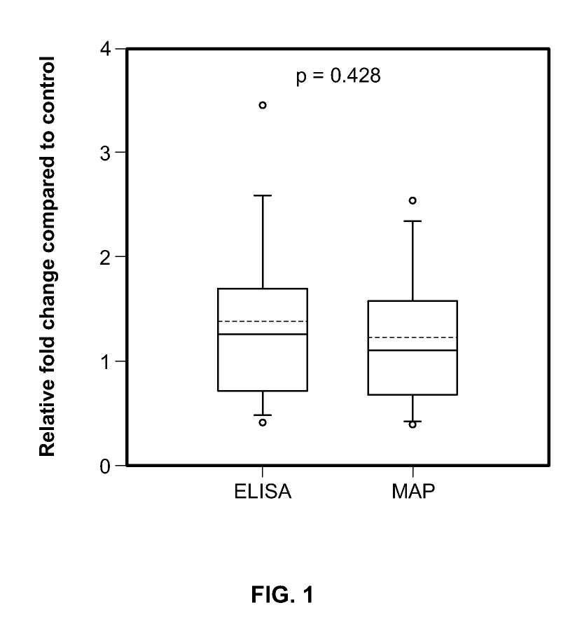

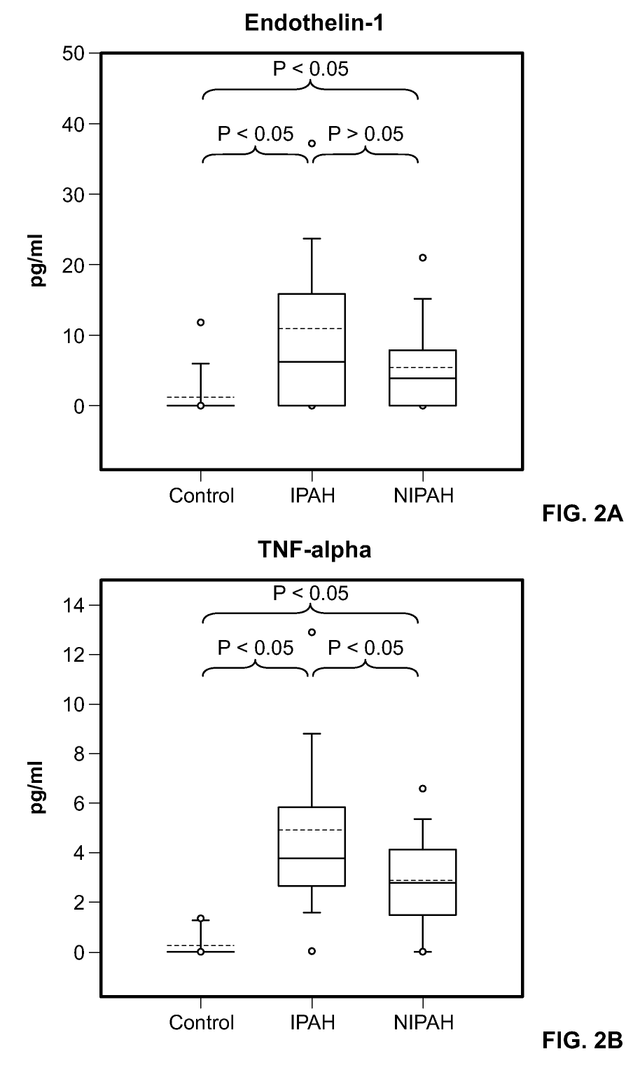

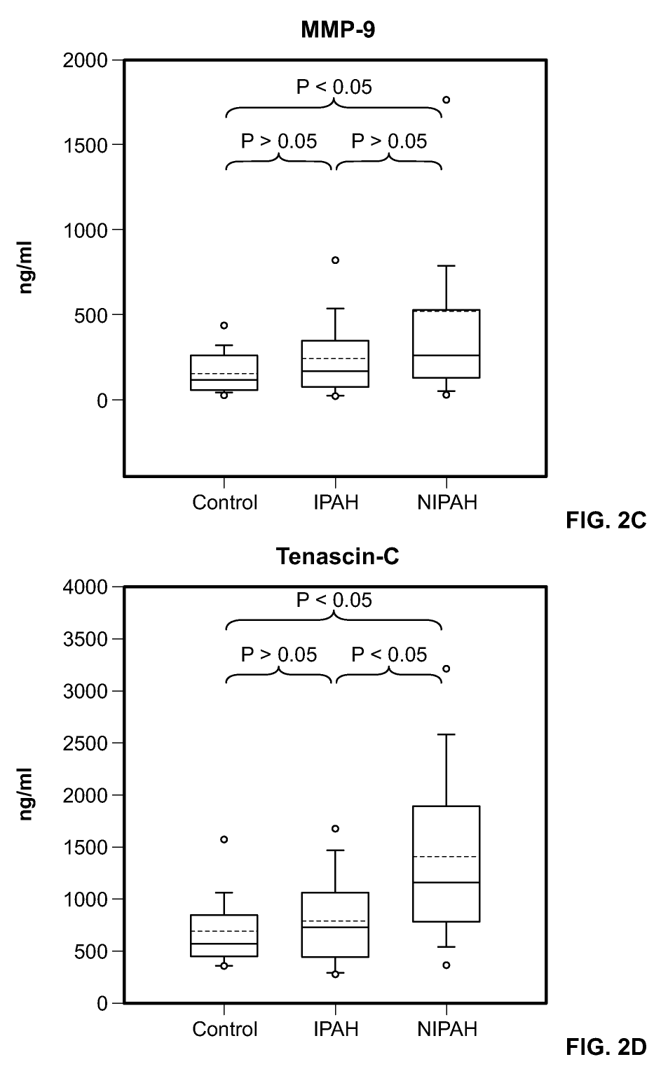

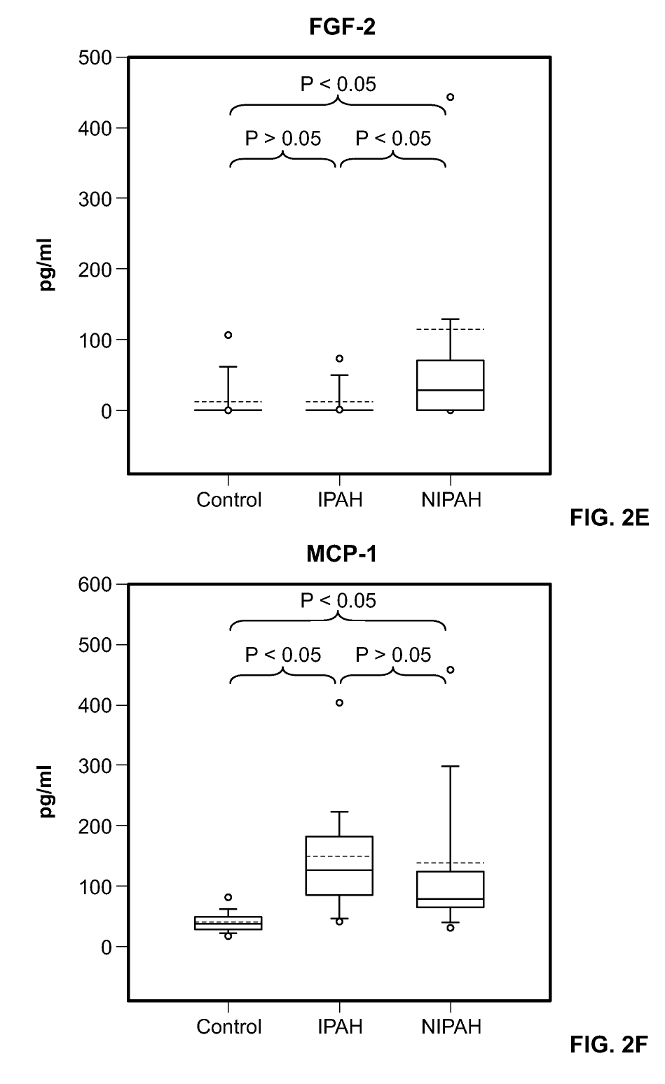

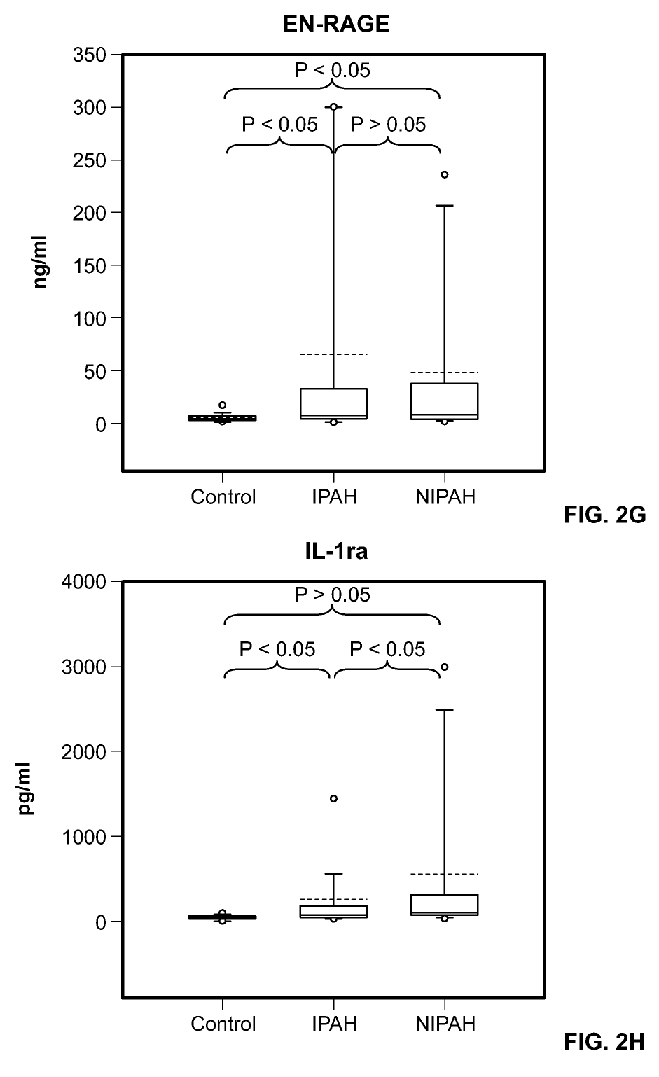

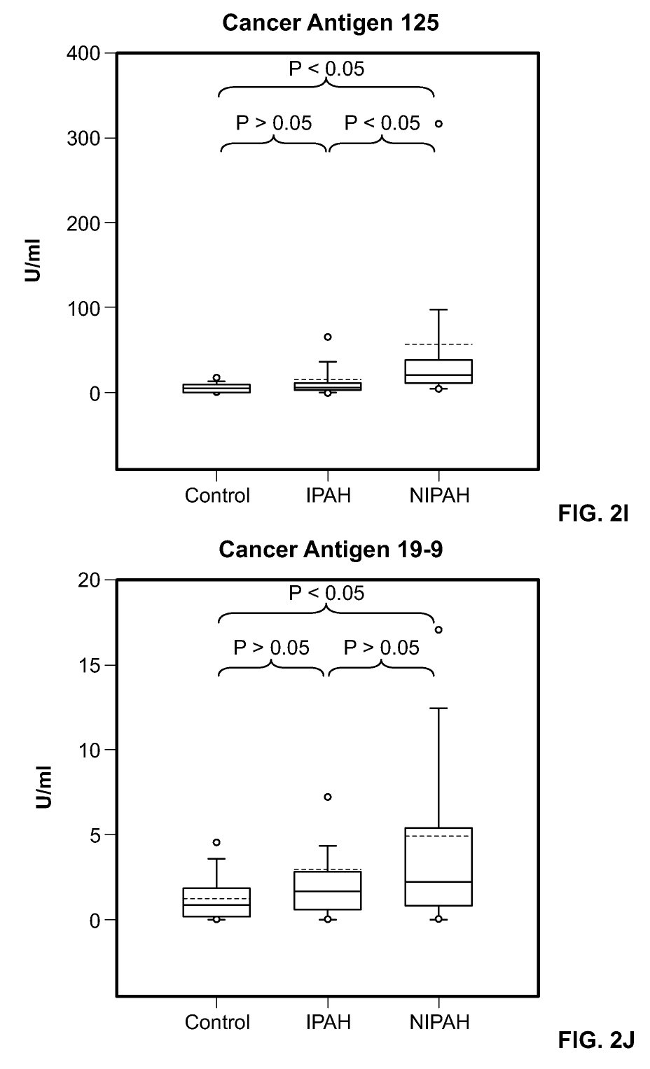

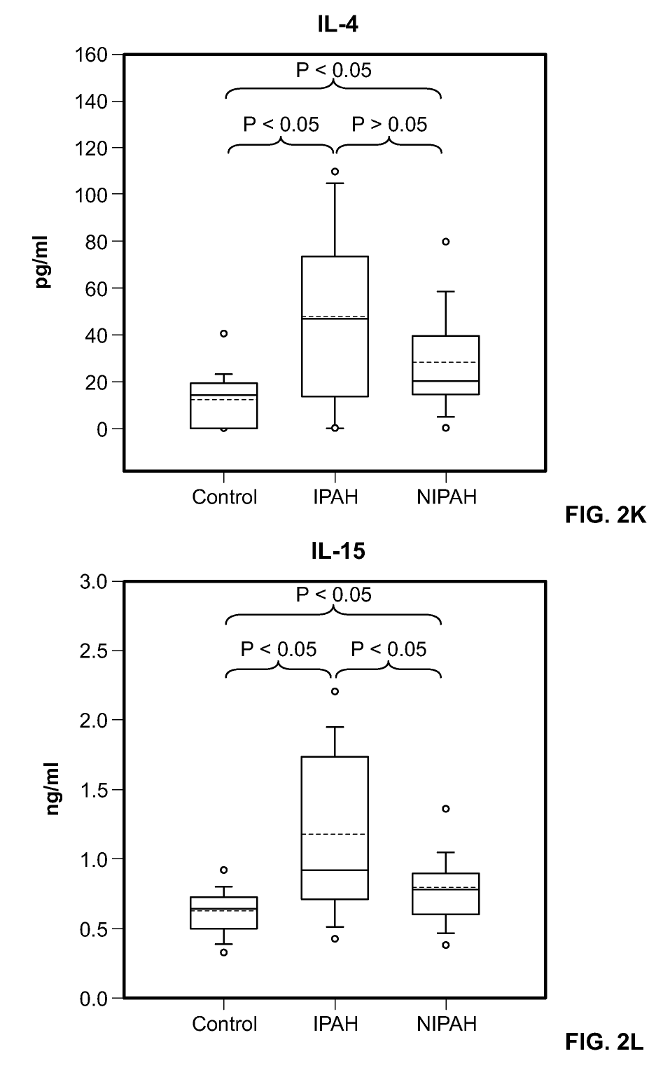

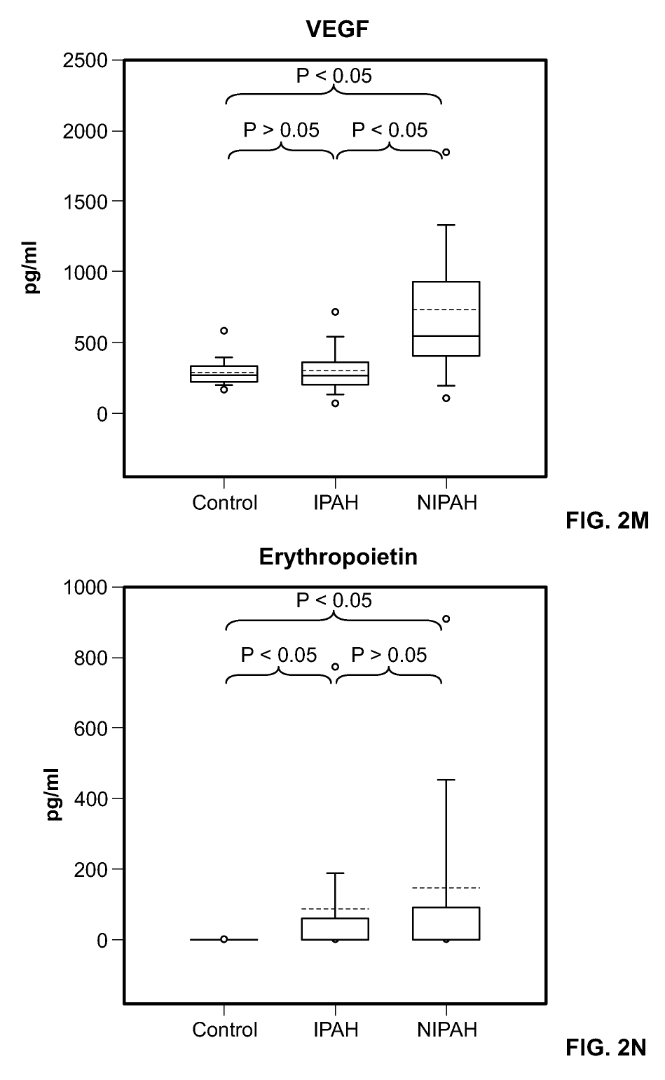

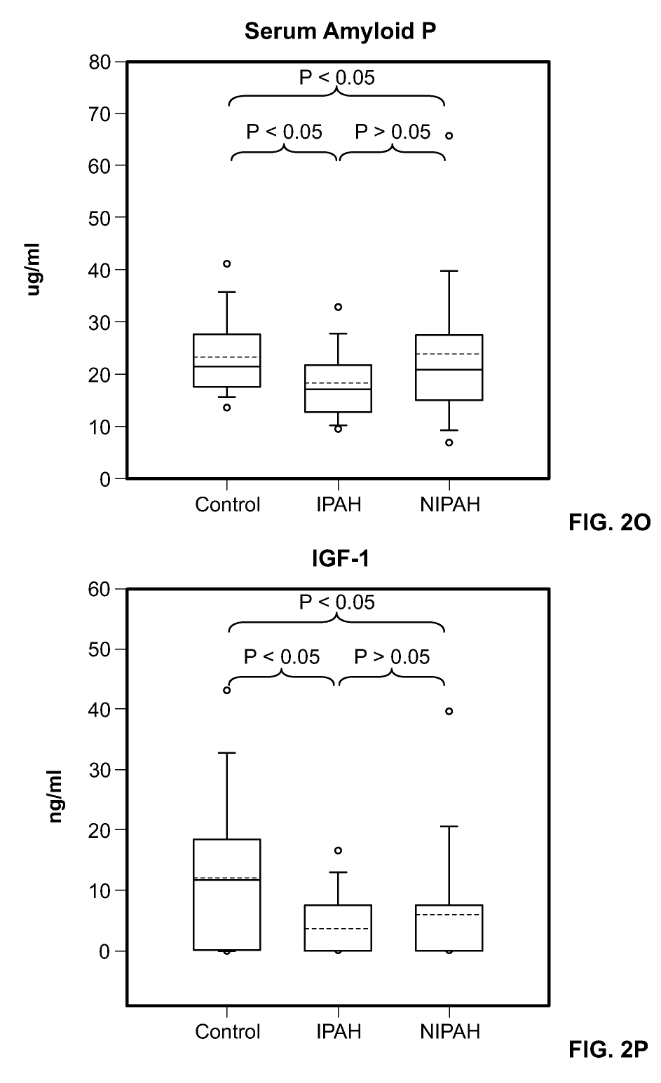

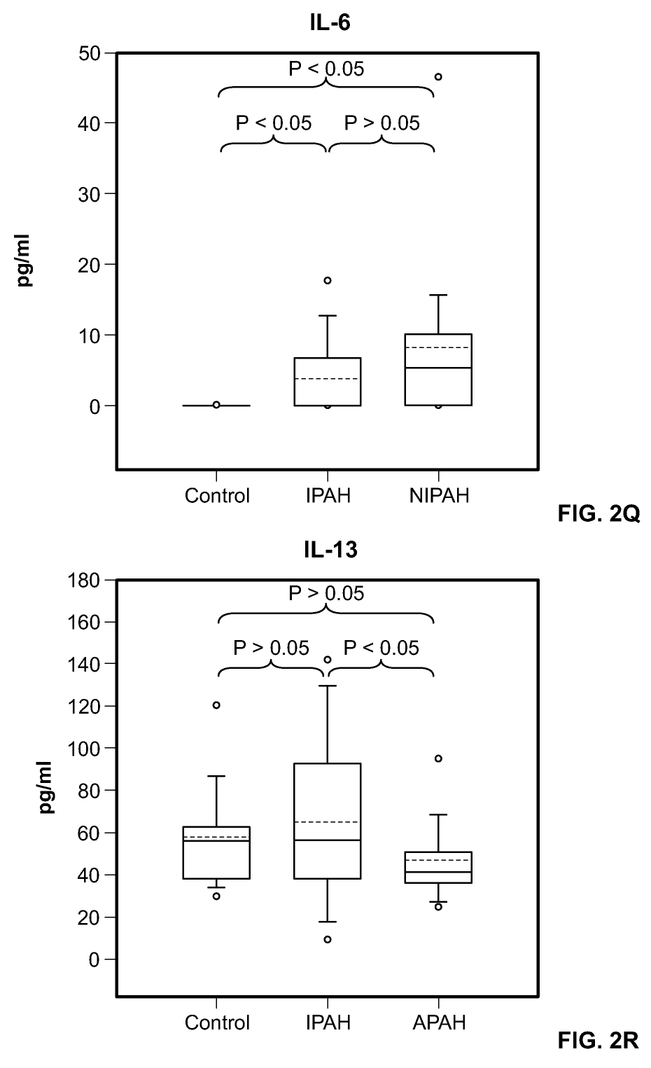

While MAP analysis represents a precise, rapid, and state-of-the-art method for quantifying the levels of specific circulating antigens using small quantities of plasma, to confirm and compare this method to another robust, more established technique a sandwich ELISA was to measure the levels of TN-C in IPAH patient plasma samples. As shown in FIG. 1, fold-increases for TN-C in IPAH patient plasma were the same using either MAP analysis or the sandwich ELISA (p=0.428).

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method for diagnosis of pulmonary hypertension, in a subject, comprising the steps of: obtaining a biological sample from said subject; and testing said biological sample to determine whether or not one or more markers is over-expressed or under-expressed in said biological sample, relative to the expression of said one or more markers in a control sample, whereby the over-expression or under-expression of said one or more markers in said biological sample indicates said pulmonary hypertension, and whereby said one or more markers comprises at least one of the markers set forth in Table 5.

2. The method of claim 1, whereby said pulmonary hypertension is pulmonary arterial hypertension (PAH).

3. The method of claim 1, whereby at least one of said one or more markers comprises a protein marker.

4. The method of claim 1, whereby at least one of said one or more markers comprises a nucleic acid marker.

5. The method of claim 1, whereby said one or more markers comprises MMP-9, tenascin-C, FGF-2, or erythropoeitin (EPO).

6. The method of claim 1, whereby the method is a multi-analyte profiling method.

7. The method of claim I, whereby the method is a high-throughput method.

8. The method of claim 1, wherein said biological sample is blood, sera, plasma, saliva, sperm, urine, mucous, cerebral spinal fluid, or any combination thereof.

9. The method of claim 1, wherein a marker expression in a biological sample that is collected from a subject is over-expressed by about 1.0 to about 147 fold relative to the expression level of the corresponding marker in a control sample.

10. The method of claim I, whereby a marker expression in a biological sample that is collected from a subject is under-expressed by about 0.1 to about 1.7 fold relative to the expression level of the corresponding marker in a control sample.

11. The method of claim 1, whereby the over-expression indicates an increased chance of being afflicted with a palmonary hypertension.

12. The method of claim 1, whereby the under-expression indicates an reduced chance of being afflicted with a palmonary hypertension.

13. A method for detecting markers associated with pulmonary hypertension, in a subject, comprising the steps of: obtaining a biological sample from said subject; and testing said biological sample to determine whether or not one or more markers is over-expressed or under-expressed in said biological sample, relative to the expression of said one or more markers in a control sample, whereby the over-expression or under-expression of said one or more markers in said biological sample indicates said pulmonary hypertension, and whereby said one or more markers comprises at least one of the markers set forth in Table 5.

14. The method of claim 13, whereby said pulmonary hypertension is pulmonary arterial hypertension (PAH).

15. The method of claim 13, whereby at least one of said one or more markers comprises a protein marker.

16. The method of claim 13, whereby at least one of said one or more markers comprises a nucleic acid marker.

17. The method of claim 13, whereby said one or more markers comprises MMP-9, tenascin-C, FGF-2, or erythropoeitin (EPO).

18. The method of claim 13, whereby the method is a multi-analyte profiling method.

19. The method of claim 13, whereby the method is a high-throughput method.

20. The method of claim 13, wherein said biological sample is blood, sera, plasma, saliva, sperm, urine, mucous, cerebral spinal fluid, or any combination thereof.

21. The method of claim 13, wherein a marker expression in a biological sample that is collected from a subject is over-expressed by about 1.0 to about 147 fold relative to the expression level of the corresponding marker in a control sample.

22. The method of claim 13, whereby a marker expression in a biological sample that is collected from a subject is under-expressed by about 0.1 to about 1.7 fold relative to the expression level of the corresponding marker in a control sample.

23. The method of claim 13, whereby the over-expression indicates an increased chance of being afflicted with a palmonary hypertension.

24. The method of claim 13, whereby the under-expression indicates an reduced chance of being afflicted with a palmonary hypertension.

25. A method for predicting the progression of pulmonary hypertension, in a subject, comprising the steps of: obtaining a biological sample from said subject; and testing said biological sample to determine whether or not one or more markers is over-expressed or under-expressed in said biological sample, relative to the expression of said one or more markers in a control sample, whereby the over-expression or under-expression of said one or more markers in said biological sample indicates the progression of said pulmonary hypertension, and whereby said one or more markers comprises at least one of the markers set forth in Table 5.

26. The method of claim 25, whereby said pulmonary hypertension is pulmonary arterial hypertension (PAH).

27. The method of claim 25, whereby at least one of said one or more markers comprises a protein marker.

28. The method of claim 25, whereby at least one of said one or more markers comprises a nucleic acid marker.

29. The method of claim 25, whereby said one or more markers comprises MMP-9, tenascin-C, FGF-2, or erythropoeitin (EPO).

30. The method of claim 25, whereby the method is a multi-analyte profiling method.

31. The method of claim 25, whereby the method is a high-throughput method.

32. The method of claim 25, wherein said biological sample is blood, sera, plasma, saliva, sperm, urine, mucous, cerebral spinal fluid, or any combination thereof.

33. The method of claim 25, wherein a marker expression in a biological sample that is collected from a subject is over-expressed by about 1.0 to about 147 fold relative to the expression level of the corresponding marker in a control sample.

34. The method of claim 25, whereby a marker expression in a biological sample that is collected from a subject is under-expressed by about 0.1 to about 1.7 fold relative to the expression level of the corresponding marker in a control sample.

35. The method of claim 25, whereby the over-expression indicates an increased chance of being afflicted with a palmonary hypertension.

36. The method of claim 25, whereby the under-expression indicates an reduced chance of being afflicted with a palmonary hypertension.

37. A method of providing prognosis for pulmonary hypertension, in a subject, comprising the steps of: obtaining a biological sample from said subject; and testing said biological sample to determine whether or not one or more markers is over-expressed or under-expressed in said biological sample, relative to the expression of said one or more markers in a control sample, whereby the over-expression or under-expression of said one or more markers in said biological sample indicates said pulmonary hypertension, and whereby said one or more markers comprises at least one of the markers set forth in Table 5.

38. A method of screening a library of compounds to identify a compound to treat pulmonary hypertension, comprising the steps of: testing a biological sample to determine whether or not one or more markers is over-expressed or under-expressed in said biological sample, relative to the expression of said one or more markers in a control sample, whereby said one or more markers comprises at least one of the markers set forth in Table 5, thereby screening said library of compounds.

39. A method of determining a therapy for pulmonary hypertension, in a subject, comprising the steps of: obtaining a biological sample from said subject; and testing said biological sample to determine whether or not one or more markers is over-expressed or under-expressed in said biological sample, relative to the expression of said one or more markers in a control sample, whereby the over-expression or under-expression of said one or more markers in said biological sample indicates said pulmonary hypertension, and whereby said one or more markers comprises at least one of the markers set forth in Table 5.

40. A method to monitor a response to a therapy for pulmonary hypertension, in a subject, comprising the steps of: obtaining a biological sample from said subject; and testing said biological sample to determine whether or not one or more markers is over-expressed or under-expressed in said biological sample, relative to the expression of said one or more markers in a control sample, whereby the over-expression or under-expression of said one or more markers in said biological sample indicates said pulmonary hypertension, and whereby said one or more markers comprises at least one of the markers set forth in Table 5.

41. A method to adjust a dosage of a therapy for pulmonary hypertension, in a subject, comprising the steps of: obtaining a biological sample from said subject; and testing said biological sample to determine whether or not one or more markers is over-expressed or under-expressed in said biological sample, relative to the expression of said one or more markers in a control sample, whereby the over-expression or under-expression of said one or more markers in said biological sample indicates said pulmonary hypertension, and whereby said one or more markers comprises at least one of the markers set forth in Table 5.
